(19) United States
(12) Patent Application Publication
(10) Pub. No.: US 2005/0265957 A1
(43) Pub. Date: Dec. 1, 2005

(54) Polymerized formamides for use in delivery of compounds to cells

(76) Inventors: Sean D. Monahan, Mazomanie, WI (US); Lisa Nader, Seattle, WA (US); Vladimir G. Budker, Middleton, WI (US); James E. Hagstrom, Middleton, WI (US); Paul M. Slattum, Madison, WI (US); Jon A. Wolff, Madison, WI (US)

Correspondence Address: MIRUS CORPORATION, 505 SOUTH ROSA RD, MADISON, WI, 53719, US

(21) Appl. No.: 11/102,432

(22) Filed: Apr. 8, 2005

Related U.S. Application Data
(60) Provisional application No. 60/560,768, filed on Apr. 8, 2004.

(51) Int. Cl. A61K 48/00

(52) U.S. Cl. 424/78.27; 514/44; 525/54.1

(57) ABSTRACT

The invention provides for polycations for condensation and delivery of polynucleotides to cells. Processes for forming the polycations by the polymerization of formamide monomers is also described.

### CROSS-REFERENCE TO RELATED APPLICATIONS

This application claims the benefit of U.S. Provisional Application No. 60/560,768, filed Apr. 8, 2004.

### FIELD OF INVENTION

The present invention relates to formulations and methods for the delivery of polynucleotides, oligonucleotides and small RNA's to cells in vitro and in vivo.

### BACKGROUND OF THE INVENTION

Gene And Nucleic Acid-Based Delivery—Gene or polynucleotide transfer to cells is an important technique for biological and medical research as well as potentially for therapeutic applications. The polynucleotide needs to be transferred across the cell membrane and into the cell. For polynucleotides encoding expressible genes, the polynucleotide must be delivered to the cell nucleus where the gene can be transcribed. Gene transfer methods currently being explored include viral vectors and non-viral methods.

Non-viral vectors are also being developed in order to transfer polynucleotides into mammalian cells. For non-viral vectors, an expressible gene is typically cloned into a plasmid. The desired gene is recombinantly inserted into polynucleotide vector along with a mammalian promoter, enhancer, or other sequences that enable the gene to be expressed in mammalian cells. Plasmid DNA can be prepared and purified from bacterial cultures. Alternatively, polynucleotides for delivery to cells can by made enzymatically such as by PCR, or they can be synthesized chemically. The polynucleotides can be incorporated into lipid vesicles (liposomes including cationic lipids such as Lipofectin) which transfer the polynucleotide into the target mammalian cell. Polynucleotides can also be complexed with polymers such as polylysine, polyethylenimine, and proteins. Other methods of polynucleotide delivery to cells include electroporation, biolistic technologies, direct injection into tissue (Wolff et al 1990) and intravascular delivery (U.S. Pat. No. 6,627,616).

Gene delivery approaches can be classified into direct and indirect methods. Some of these gene transfer methods are most effective when directly injected into a tissue space. Direct methods using many of the above gene transfer techniques are being used to target tumors, muscle, liver, lung, and brain. Other methods are most effective when applied to cells or tissues that have been removed from the body. Following this treatment the genetically-modified cells are then transplanted back into the body.

Gene Therapy And Nucleic Acid-Based Therapies—With gene therapy, a disease state can be directly treated by inserting a corrective polynucleotide into cells. In contrast, traditional drug based approaches act downstream on the products of the genes (proteins, enzymes, enzyme substrates and enzyme products). Although, the initial motivation for gene therapy was the treatment of genetic disorders, it is becoming increasingly apparent that gene therapy has the potential to be useful in the treatment of a broad range of acquired diseases such as cancer, infectious diseases, heart disease, arthritis, and neurodegenerative disorders (such as Parkinson's and Alzheimer's).

In addition to providing an exogenous gene, gene therapy also has the potential to inhibit endogenous genes. Several mechanisms exist for specifically inhibiting expression of an endogenous gene. These include antisense nucleic acid, ribozymes, and small inhibitory RNA (siRNA) mediated RNA interference (RNAi). Antisense inhibition involves single stranded polynucleotide that is complementary to the target mRNA. Ribozymes are catalytic RNAs capable of specifically cleaving a target mRNA. SiRNAs are short double stranded RNAs that are identical in sequence to a segment of the expressed target gene and, in conjunction with cellular proteins, cause the degradation of the target RNA.

Gene transfer can also be used as a vaccination against infectious diseases and cancer. When a foreign gene is transferred to a cell and expressed, the resultant protein is presented to the immune system. Expression of the viral gene within a cell simulates a viral infection without the danger of an actual viral infection and induces a more effective immune response. This approach may be more effective in for fighting latent viral infections such as human immunodeficiency virus, Herpes and cytomegalovirus.

Polymers for Polynucleotide Delivery—Polymers have been used in research for the delivery of polynucleotides to cells. One of the several methods of polynucleotide delivery to cells is the use of polynucleotides/polycation complexes. It has been shown that cationic proteins, like histones and protamines, or synthetic polymers, like polylysine, polyarginine, polyornithine, DEAE dextran, polybrene, and polyethylenimine may be effective intracellular polynucleotide delivery agents. The following are some important principles involving the mechanism by which polycations facilitate uptake of polynucleotides:

Polycations facilitate nucleic acid condensation. The volume which one polynucleotide molecule occupies in a complex with polycations is drastically lower than the volume of the free polynucleotide molecule. The size of a polynucleotides/polymer complex is probably critical for gene delivery in vivo and possible for in vitro as well. For intravascular delivery, the polynucleotide needs to cross the endothelial barrier in order to reach the parenchymal cells of interest. The largest endothelial fenestrae (holes in the endothelial barrier) occur in the liver and have an average diameter of 100 nm. The trans-epithelial pores in other organs are much smaller. For example, muscle endothelium can be described as a structure that has a large number of small pores with a radius of 4 nm, and a very low number of large pores with a radius of 20-30 nm. The hydrodymanic in vivo delivery process (U.S. Pat. No. 6,627,616), however, is thought to transiently increase the sizes of pores in the vascular endothelium. The size of the polynucleotide complexes is also important for the cellular uptake process. After binding to the cells the polynucleotide/polycation complex is likely taken up by endocytosis. Since endocytic vesicles have a typical internal diameter of about 100 nm, polynucleotide complexes smaller than 100 nm are preferred.

Polycations may provide attachment of polynucleotides to the cell surface. The polymer forms a cross-bridge between the polyanionic polynucleotide and the polyanionic surface of a cell. As a result, the mechanism of polynucleotide translocation to the intracellular space might be non-specific adsorptive endocytosis. Furthermore, polycations provide a convenient linker for attaching specific ligands to the complex. The polynucleotide/polycation complexes could then be targeted to specific cell surface receptors or cell types.

The polynucleotides in polycation complexes are protected against nuclease degradation. This protection is important for both extra- and intracellular preservation of polynucleotide since nucleases are present in serum and endosomes/lysosomes. Protection from degradation in endosomes/lysosomes is enhanced by preventing organelle acidification. Some polymers, such as polyethylenimine or polypropylacrylic acid, may disrupt endosomallysosomal acidification and/or disrupt cellular membranes, thereby enhancing delivery. Disruption of endosomal/lysosomal function has also been accomplished by linking endosomal or membrane disruptive agents such as fusion peptides or adenoviruses to the polycation or complex.

Condensation of nucleic acid—A significant number of multivalent cations with widely different molecular structures have been shown to induce condensation of nucleic acid. Multivalent cations with a charge of three or higher have been shown to condense DNA. These include spermidine, spermine, Co(NH₃)₆³⁺, Fe³⁺, and natural or synthetic polymers such as histone H1, protamine, polylysine, and polyethylenimine. Analysis has shown DNA condensation to be favored when 90% or more of the charges along the sugar-phosphate backbone are neutralized. Neutral and anionic polymers can increase repulsion between DNA and its surroundings, therefore compacting the DNA. Most significantly, spontaneous DNA self-assembly and aggregation processes have been shown to result from the confinement of large amounts of DNA due to excluded volume effect.

The mechanism of polynucleotide condensation is not clear. The electrostatic force between unperturbed helices arises primarily from a counter-ion fluctuation mechanism requiring multivalent cations and plays a major role in polynucleotide condensation. The hydration forces predominate over electrostatic forces when the nucleic acid helices approach closer then a few water diameters. The electrophoretic mobility of polynucleotide/polycation complexes can change from negative to positive in excess polycation.

Surface charging—As discussed previously, polycations can help the polynucleotide complexes to adhere to a cell surface. However, negative surface charge would be more desirable for many practical applications, i.e. in vivo delivery. The phenomenon of surface recharging is well known in colloid chemistry and has been described for non-viral polynucleotide complexes (U.S. Pat. No. 6,383,811).

The Use of pH-Sensitive Lipids, Amphipathic Compounds, and Liposomes for Nucleic Acid Delivery—Cationic liposomes may deliver DNA either directly across the plasma membrane or via the endosome compartment. Regardless of its exact entry point, much of the DNA within cationic liposomes accumulates in the endosome compartment. Several approaches have been investigated to prevent loss of the foreign DNA in the endosomal compartment by protecting it from hydrolytic digestion or enabling its escape into the cytoplasm. Viruses and viral fusion peptides as well as membrane active compounds have been included to disrupt endosomes or promote fusion of liposomes with endosomes and facilitate release of DNA into the cytoplasm (Kamata et al. 1994; Wagner et al. 1994).

The Use of pH-Sensitive Polymers for Nucleic Acid Delivery—Polymers that are pH-sensitive have found broad application in the area of drug delivery because of their ability to exploit various physiological and intracellular pH gradients for the purpose of controlled release of drugs. pH sensitivity can be broadly defined as any change in polymer's physico-chemical properties over a range of pH. Narrower definitions demand significant changes in the polymer's ability to retain or release a bioactive substance in a physiologically tolerated pH range (typically pH 5.5-8). All polyions can be divided into three categories based on their ability to donate or accept protons in aqueous solutions: polyacids, polybases and polyampholytes. Use of pH-sensitive polyacids in drug delivery applications usually relies on their ability to a) become soluble with a pH increase (acid/salt conversion), b) form a complex with other polymers over a change of pH, or c) undergo significant change in hydrophobicity/hydrophilicity balance. Combinations of all three above factors are also possible.

Delivery of siRNA—Recently, there has been a great deal of research interest in the delivery of RNA oligonucleotides to cells due to the discovery of RNA interference (RNAi). RNAi interference results in the knockdown of protein production within cells, via the interference of the small interfering RNA (siRNA) with the mRNA involved in protein production. This interference therefore curtails gene expression. The delivery of small double stranded RNAs (small interfering RNAs, or siRNAs, and microRNAs) to cells, has resulted in a greater than 80% knockdown of endogenous gene expression levels within the cell. Additionally, through the use of specific siRNAs, gene knockdown can be accomplished without inhibiting the expression of non-targeted genes.

### SUMMARY OF THE INVENTION

In a preferred embodiment, we describe the use of polymers resulting from the polymerization of formamides for the delivery of biological materials to cells. The polymer is prepared from the polymerization of the formamide imidate (formed upon the treatment of the formamide with acid) in solution together with the formamide at ambient temperatures to form polymerized formamide. The resulting polymers are cationic based upon the ability to condense polyanions (such as poly acrylic acid and DNA). The polymers can be homopolymers from the polymerization of one monomeric formamide. Alternatively, the polymers can be heteropolymers or copolymers derived from the polymerization of two or more monomeric formamides. These resulting cationic polymers condense polynucleotides to form complexes that can then be delivered both in vitro and in vivo.

In a preferred embodiment we describe a cationic polymer that is susceptible to cleavage under basic conditions. This cleavability may be adjusted depending on the formamide employed in the polymerization reaction or ratios or different formamides utilized in the polymerization reaction to form copolymers. The cleavability may also be adjusted by other components in the complex solution such as salt, recharging with another polymer, surfactant, lipid, peptide or targeting agent.

In a preferred embodiment we describe an in vivo process for delivering a polynucleotide to a cell comprising: polymerizing one or more formamides, associating the polynucleotide with the polymerized formamide in an aqueous solution to form a complex, and bringing the complex into contact with the cell. In another embodiment, the complex is more stable if 150 mM salt is added to the complex. The complex may also be formed in the presence of salt. A stable particle comprises condensed polynucleotide wherein the size of the complex does not rapidly increase nor does the polynucleotide rapidly decondense if the complex is exposed to salt at physiological concentrations. Bringing the complex into contact with the cell may comprise: directly injecting the complex in an aqueous solution into a tissue or inserting the complex in an aqueous solution into a vessel in a mammal for delivery to cell in a tissue to which the vessel either supplies or drains a bodily fluid.

In a preferred embodiment we describe in vivo process for delivering a polynucleotide to a cell comprising polymerizing one or more formamides, association of the polynucleotide with polymerized formamide in an organic solution to form a complex, and bringing the complex into contact with the cell. Bringing the complex into contact with the cell may comprise: directly injecting the complex in an organic solution into a tissue or mixing the complex in an organic solution with an aqueous solution and injecting into a vessel in a mammal for delivery to cell in a tissue to which the vessel either supplies or drains a bodily fluid. In a preferred embodiment we describe an in vitro process for delivering a polynucleotide to a cell comprising: associating the polynucleotide with polymerized formamide in an aqueous or organic solution to form a complex and contacting the cell with the complex.

In a preferred embodiment, we describe a process for delivering a polynucleotide to a cell comprising: associating a polynucleotide with a polymerized formamide to form a binary complex, associating the binary complex with an polyanion of amphipathic compound to form a ternary complex, and associating the ternary complex with a cell. The amphipathic compound may be selected from the list comprising: polymers, peptides, targeting groups, steric stabilizers, surfactants and lipids. The amphipathic compound may be cationic, anionic, neutral, or zwitterionic. The resultant ternary complex can have a net surface charge that is positive, negative or neutral. The amphipathic compound may also be modified to contain one or more functional groups that increase transfection efficiency. The amphipathic compound may be modified prior to ternary complex formation of after ternary complex formation. The ternary complex may be delivered to a cell in vivo or in vitro.

Further objects, features, and advantages of the invention will be apparent from the following detailed description when taken in conjunction with the accompanying drawings.

### BRIEF DESCRIPTION OF THE DRAWINGS

FIG. 1. Graph illustrating inhibition of firefly luciferase activity in hepatocytes following in vivo delivery of Luciferase specific siRNA (Complexes II, IV and VI). Delivery of no siRNA or EGFP control siRNA (Complexes I, III, V and VI) show no inhibition of firefly luciferase activity. Complexes II and III were naked polynucleotide delivered by hydrodynamic tail vein injection. Complexes IV-VII were recharged siRNA/poly(formamide) complexes delivered by hydrodynamic tail vein injection.

FIG. 2 Graph illustrating siRNA delivery to cells in vitro using siRNA/poly(formamide) (MC1015) complexes.

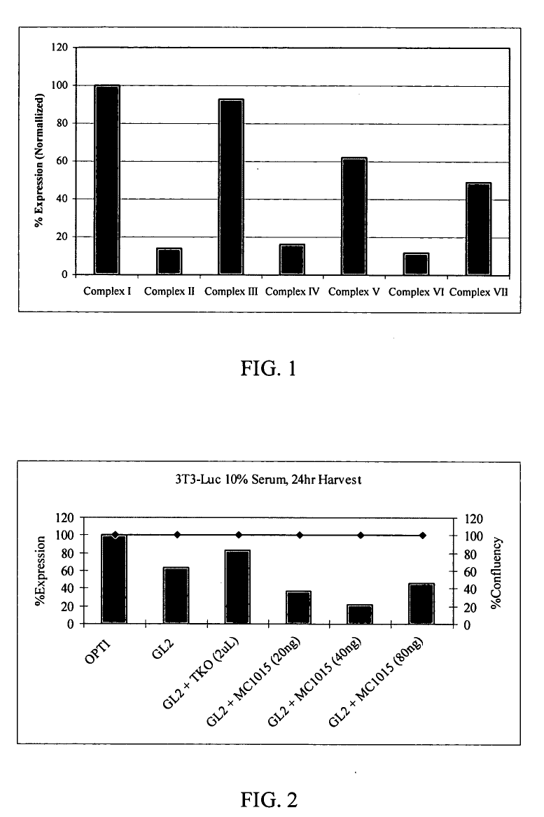

### DETAILED DESCRIPTION OF THE INVENTION

We describe the formation of a polycation from the polymerization of the imidate derived from a formamide. For example, we have prepared a polycation from polymerization of the dimethylformamide imidate (formed upon the treatment of dimethylformamide (DMF) with hydrochloric acid (HCl gas or HCl in diethylether)). The dimethylformamide imidate can, but does not need to be, isolated for use in the invention. The polymer may be prepared from the polymerization of the dimethylformamide imidate (formed upon the treatment of the dimethylformamide with HCl gas or HCl in diethylether) in solution together with the dimethylformamide at ambient temperatures to form poly-DMF (MC105). That polymer has formed can be tested by monitoring the extent of fluorescence quenching of a labeled polyanion, (e.g., rhodamine labeled pDNA in the presence of the polymer (Trubetskoy et al. 1999). The polymer can be used to complex with DNA for delivery of the DNA to a cell.

Particle sizing and fluorescence condensation (quenching) experiments of pDNA with poly-DMF have been conducted in several aqueous solutions at acidic pH. Aqueous solutions include H₂O, 25 mM Hepes (pKₐ 7.55), and 25 mM Citrate (pKₐ 3.1). These studies indicate strong condensation of pDNA by the polymer, with particle sizes of approximately 24 nm to 80 nm. The pDNA is rapidly decondensed when the pH of the solution is raised.

When pDNA is condensed with poly-DMF in H₂O, the resulting solution has an observed pH. Destruction of particles when the pH of the solution is increased is observed both by fluorescence decondensation and the loss of particles by particle sizing. The pH of the resulting solution is then observed to decrease over time until finally stabilizing. This drop in pH of the solution supports the degradation of the polymer to DMF monomers, as the regeneration of the reaction starting material (DMF) would yield an equivalent of HCl. Comparatively, the addition of unpolymerized imidate to DNA under the same conditions, yield the same observed pH but without condensation of the pDNA.

Fluorescence condensation and the stability of the particles based on fluorescence quenching were further investigated in 150 mM NaCl (physiological concentration). In 150 mM NaCl, polyDMF strongly condenses pDNA. In contrast to polyDMF/DNA particles formed in no salt, the particles in salt are stable (no increased fluorescence is observed) for greater than 4 hrs when exposed to basic conditions (by the addition of dilute NaOH, final pH 8.5). Particles formulated in 150 mM NaCl and recharged with a polyanion were also found to remain stable under the described basic conditions.

We have shown the utility of poly-formamide polymers for the condensation and delivery, in vitro and in vivo, of both pDNA and siRNA. The resulting particles are salt (150 mM NaCl) stable and can be recharged to control the overall charge of a particle. Recharged particles in general tend to stay in circulation longer than positively charged particles and are attractive for in vivo delivery in some cases. Recharged formulations included a variety of polyanions, including but not limited to: polyacrylic acid (20 kDa, Sigma Chemical Company) and poly-succinylated-L-lysine (30 kDa, Sigma Chemical Company). Complexes have been delivered in vivo to the liver (via the tail vein, bile duct, portal vein, and hepatic vein), lung (via the tail vein), spleen (via the tail vein), kidney (via the tail vein), heart (via the tail vein) and bladder (via bladder injection). Other potential sites for delivery include but are not limited to the stomach, intestine, joints, muscle, eye, and lymphatic system.

A number of formamides can be utilized for this type of polymerization. For example, poly-dibutylformamide was synthesized by forming the formamide imidate via bubbling HCl (g) through a cooled, neat, solution. N,N-dibutylformamide was not completely converted to the imidate, as some solution remained, together with precipitated imidate. After allowing the reaction to warm to room temperature, the imidate melted, initiating the polymerization.

There are known methods in the art for modifying formamides on the nitrogen atom. These methods make it possible to place a variety of substituents on the formamide nitrogen atom. The modified formamides may be used in the polymerization reaction to afford a large variety of polymers. Using this methodology, both homogeneous polymers (polymerization of one type of monomeric formamide) and copolymers (polymerization of more than one type of monomeric formamides) can be prepared.

Modification of the formamide on the nitrogen atom may include but is not limited to alkyl groups. Additionally, substitution can be, but does not have to be, symmetrical, (for example, an ethyl and a butyl group). These groups also may possess different degrees of saturation. Other modifications of the formamide include the addition of other functional groups (including but not limited to, imidazoles, amines, and carbonyl compounds) linked off the nitrogen atom prior to polymerization. These functional groups may then be modified with or interacted with substances such as targeting agents, stabilizing agents, peptides, DNA, RNA, drugs and other cargo (both prior to and following polymerization).

It may be possible for post synthetic modification on the nitrogen atom following the polymerization reaction. There are known methods in the art for modification of amines. Modifications include but are not limited to, alkylation, acylation and reductive amination. These methods make it possible to introduce groups selected from the group comprising: steric stabilizers, fluorescent labels, hydrophobic groups, interaction modifiers, and targeting groups. These groups are meant to affect or enhance the delivery of complexes to cells and organs.

Lability, as well as several other properties, of the polymer may be affected by the substitution on the nitrogen. These properties may include the hydrophilic/hydrophobic character, polymer packing, overall size (length of polymer) and the interaction between the polymer and the cargo. It may be possible to dial in modifications that are desired to deliver the cargo of interest.

In addition to formamides, related systems in which the formamide oxygen atom has been replaced by another heteroatom are encompassed within this invention. For example, we describe the polymerization of N,N-Dimethylthioformamide to form a polycation that is capable of condensing polyanions, for example pDNA.

Polymerized formamides and similar polymer systems described herein may have many uses as a delivery agent. They may have the potential to deliver not only DNA and siRNA but also drugs and other cargo. The cationic nature of this polymer allows for it to interact electrostatically (noncovalently) with negatively charged cargo. The polymer is labile, appears to have low toxicity in vivo (bile duct) and may be applicable for many routes of delivery enabling increased drug delivery.

Definitions:

Polymerizedformamide—A polymerized formamide is the polymer resulting from the polymerization of the imidate of a formamide (formed from the addition of acid (HX) to the formamide) or the polymerization of the imidate of the formamide in which the formamide oxygen atom is replaced by another heteroatom (R₃=heteroatom, for example a thioformamide R₃=S). The polymerized formamide may be a heteropolymer or a copolymer. The a polymerized formamide is not meant to include polymers arising from the polymerization of a functional group on a formamide resulting in a polymer with two or more formamide groups as substituents. 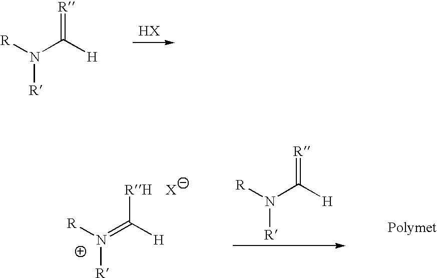
R and R′ can independently be hydrogen (H), a primary, secondary, or tertiary carbon in which the substitution is not limited, a methane group in which the substitution is not limited, a methyne group in which the substitution is not limited, or as part of an aromatic or heteroring system. The carbon atom can not have a double bond to an oxygen atom (i.e. be a carbonyl group). R″ can be a heteroatom.

Complex—Two molecules are combined to form a complex through a process called complexation, or complex formation, if they are in contact with one another through noncovalent interactions such as electrostatic interactions, hydrogen bonding interactions, or hydrophobic interactions.

Binary complex—A binary complex is meant to include the complex formed between a polynucleotide and an RNA with a poly(aminomethylene) glycol.

Ternary complex—A ternary complex is the complex formed when one or more components is added to a binary complex.

Polynucleotide—The term polynucleotide, or nucleic acid or polynucleic acid, is a term of art that refers to a polymer containing at least two nucleotides. Nucleotides are the monomeric units of polynucleotide polymers. Polynucleotides with less than 120 monomeric units are often called oligonucleotides. Natural nucleic acids have a deoxyribose- or ribose-phosphate backbone. An artificial or synthetic polynucleotide is any polynucleotide that is polymerized in vitro or in a cell free system and contains the same or similar bases but may contain a backbone of a type other than the natural ribose-phosphate backbone. These backbones include: PNAs (peptide nucleic acids), phosphorothioates, phosphorodiamidates, morpholinos, and other variants of the phosphate backbone of native nucleic acids. Bases include purines and pyrimidines, which further include the natural compounds adenine, thymine, guanine, cytosine, uracil, inosine, and natural analogs. Synthetic derivatives of purines and pyrimidines include, but are not limited to, modifications which place new reactive groups such as, but not limited to, amines, alcohols, thiols, carboxylates, and alkylhalides. The term base encompasses any of the known base analogs of DNA and RNA including, but not limited to, 4-acetylcytosine, 8-hydroxy-N-6-methyladenosine, aziridinylcytosine, pseudoisocytosine, 5-(carboxyhydroxylmethyl) uracil, 5-fluorouracil, 5-bromouracil, 5-carboxymethylaminomethyl-2-thiouracil, 5-carboxymethylaminomethyluracil, dihydrouracil, inosine, N6-isopentenyladenine, 1-methyladenine, 1-methylpseudo-uracil, 1-methylguanine, 1-methylinosine, 2,2-dimethyl-guanine, 2-methyladenine, 2-methylguanine, 3-methyl-cytosine, 5-methylcytosine, N6-methyladenine, 7-methylguanine, 5-methylaminomethyluracil, 5-methoxy-amino-methyl-2-thiouracil, beta-D-mannosylqueosine, 5′-methoxycarbonylmethyluracil, 5-methoxyuracil, 2-methylthio-N-6-isopentenyladenine, uracil-5-oxyacetic acid methylester, uracil-5-oxyacetic acid, oxybutoxosine, pseudouracil, queosine, 2-thiocytosine, 5-methyl-2-thiouracil, 2-thiouracil, 4-thiouracil, 5-methyluracil, N-uracil-5-oxyacetic acid methylester, uracil-5-oxyacetic acid, pseudouracil, queosine, 2-thiocytosine, and 2,6-diaminopurine. The term polynucleotide includes deoxyribonucleic acid (DNA) and ribonucleic acid (RNA) and combinations on DNA, RNA and other natural and synthetic nucleotides.

DNA may be in form of cDNA, in vitro polymerized DNA, plasmid DNA, parts of a plasmid DNA, genetic material derived from a virus, linear DNA, vectors (P1, PAC, BAC, YAC, artificial chromosomes), expression cassettes, chimeric sequences, recombinant DNA, chromosomal DNA, an oligonucleotide, anti-sense DNA, or derivatives of these groups. RNA may be in the form of oligonucleotide RNA, tRNA (transfer RNA), snRNA (small nuclear RNA), rRNA (ribosomal RNA), mRNA (messenger RNA), in vitro polymerized RNA, recombinant RNA, chimeric sequences, anti-sense RNA, siRNA (small interfering RNA), ribozymes, or derivatives of these groups. An anti-sense polynucleotide is a polynucleotide that interferes with the function of DNA and/or RNA. Antisense polynucleotides include, but are not limited to: morpholinos, 2′-O-methyl polynucleotides, DNA, RNA and the like. SiRNA comprises a double stranded structure typically containing 15-50 base pairs and preferably 21-25 base pairs and having a nucleotide sequence identical or nearly identical to an expressed target gene or RNA within the cell. Interference may result in suppression of expression. The polynucleotide can be a sequence whose presence or expression in a cell alters the expression or function of cellular genes or RNA. In addition, DNA and RNA may be single, double, triple, or quadruple stranded. Double, triple, and quadruple stranded polynucleotide may contain both RNA and DNA or other combinations of natural and/or synthetic nucleic acids.

A delivered polynucleotide can stay within the cytoplasm or nucleus apart from the endogenous genetic material. Alternatively, DNA can recombine with (become a part of) the endogenous genetic material. Recombination can cause DNA to be inserted into chromosomal DNA by either homologous or non-homologous recombination.

A polynucleotide can be delivered to a cell to express an exogenous nucleotide sequence, to inhibit, eliminate, augment, or alter expression of an endogenous nucleotide sequence, or to affect a specific physiological characteristic not naturally associated with the cell. Polynucleotides may contain an expression cassette coded to express a whole or partial protein, or RNA. An expression cassette refers to a natural or recombinantly produced polynucleotide that is capable of expressing a gene(s). The term recombinant as used herein refers to a polynucleotide molecule that is comprised of segments of polynucleotide joined together by means of molecular biological techniques. The cassette contains the coding region of the gene of interest along with any other sequences that affect expression of the gene. A DNA expression cassette typically includes a promoter (allowing transcription initiation), and a sequence encoding one or more proteins. Optionally, the expression cassette may include, but is not limited to, transcriptional enhancers, non-coding sequences, splicing signals, transcription termination signals, and polyadenylation signals. An RNA expression cassette typically includes a translation initiation codon (allowing translation initiation), and a sequence encoding one or more proteins. Optionally, the expression cassette may include, but is not limited to, translation termination signals, a polyadenosine sequence, internal ribosome entry sites (IRES), and non-coding sequences.

The polynucleotide may contain sequences that do not serve a specific function in the target cell but are used in the generation of the polynucleotide. Such sequences include, but are not limited to, sequences required for replication or selection of the polynucleotide in a host organism.

A polynucleotide can be used to modify the genomic or extrachromosomal DNA sequences. This can be achieved by delivering a polynucleotide that is expressed. Alternatively, the polynucleotide can effect a change in the DNA or RNA sequence of the target cell. This can be achieved by hybridization, multistrand polynucleotide formation, homologous recombination, gene conversion, or other yet to be described mechanisms.

The term gene generally refers to a polynucleotide sequence that comprises coding sequences necessary for the production of a therapeutic polynucleotide (e.g., ribozyme) or a polypeptide or precursor. The polypeptide can be encoded by a full length coding sequence or by any portion of the coding sequence so long as the desired activity or functional properties (e.g., enzymatic activity, ligand binding, signal transduction) of the full-length polypeptide or fragment are retained. The term also encompasses the coding region of a gene and the including sequences located adjacent to the coding region on both the 5′ and 3′ ends for a distance of about 1 kb or more on either end such that the gene corresponds to the length of the full-length mRNA. The sequences that are located 5′ of the coding region and which are present on the mRNA are referred to as 5′ untranslated sequences. The sequences that are located 3′ or downstream of the coding region and which are present on the mRNA are referred to as 3′ untranslated sequences. The term gene encompasses both cDNA and genomic forms of a gene. A genomic form or clone of a gene contains the coding region interrupted with non-coding sequences termed introns, intervening regions or intervening sequences. Introns are segments of a gene that are transcribed into nuclear RNA. Introns may contain regulatory elements such as enhancers. Introns are removed or spliced out from the nuclear or primary transcript; introns therefore are absent in the messenger RNA (mRNA) transcript. The mRNA functions during translation to specify the sequence or order of amino acids in a nascent polypeptide. The term non-coding sequences also refers to other regions of a genomic form of a gene including, but not limited to, promoters, enhancers, transcription factor binding sites, polyadenylation signals, internal ribosome entry sites, silencers, insulating sequences, matrix attachment regions. These sequences may be present close to the coding region of the gene (within 10,000 nucleotide) or at distant sites (more than 10,000 nucleotides). These non-coding sequences influence the level or rate of transcription and translation of the gene. Covalent modification of a gene may influence the rate of transcription (e.g., methylation of genomic DNA), the stability of mRNA (e.g., length of the 3′ polyadenosine tail), rate of translation (e.g., 5′ cap), nucleic acid repair, and immunogenicity. One example of covalent modification of nucleic acid involves the action of LabelIT reagents (Mirus Corporation, Madison, Wis.).

As used herein, the term gene expression refers to the process of converting genetic information encoded in a gene into RNA (e.g., mRNA, rRNA, tRNA, or snRNA) through transcription of a deoxyribonucleic gene (e.g., via the enzymatic action of an RNA polymerase), and for protein encoding genes, into protein through translation of mRNA. Gene expression can be regulated at many stages in the process. Up-regulation or activation refers to regulation that increases the production of gene expression products (i.e., RNA or protein), while down-regulation or repression refers to regulation that decrease production. Molecules (e.g., transcription factors) that are involved in up-regulation or down-regulation are often called activators and repressors, respectively.

An RNA function inhibitor comprises any polynucleotide or nucleic acid analog containing a sequence whose presence or expression in a cell causes the degradation of or inhibits the function or translation of a specific cellular RNA, usually an mRNA, in a sequence-specific manner. Inhibition of RNA can thus effectively inhibit expression of a gene from which the RNA is transcribed. RNA function inhibitors are selected from the group comprising: siRNA, interfering RNA or RNAi, dsRNA, RNA Polymerase III transcribed DNAs encoding siRNA or antisense genes, ribozymes, and antisense nucleic acid, which may be RNA, DNA, or artificial nucleic acid. SiRNA comprises a double stranded structure typically containing 15-50 base pairs and preferably 21-25 base pairs and having a nucleotide sequence identical or nearly identical to an expressed target gene or RNA within the cell. Antisense polynucleotides include, but are not limited to: morpholinos, 2′-O-methyl polynucleotides, DNA, RNA and the like. RNA polymerase III transcribed DNAs contain promoters, such as the U6 promoter. These DNAs can be transcribed to produce small hairpin RNAs in the cell that can function as siRNA or linear RNAs that can function as antisense RNA. The RNA function inhibitor may be polymerized in vitro, recombinant RNA, contain chimeric sequences, or derivatives of these groups. The RNA function inhibitor may contain ribonucleotides, deoxyribonucleotides, synthetic nucleotides, or any suitable combination such that the target RNA and/or gene is inhibited. In addition, these forms of nucleic acid may be single, double, triple, or quadruple stranded.

Transfection—The process of delivering a polynucleotide to a cell has been commonly termed transfection or the process of transfecting and also it has been termed transformation. The term transfecting as used herein refers to the introduction of a polynucleotide or other biologically active compound into cells. The polynucleotide may be used for research purposes or to produce a change in a cell that can be therapeutic. The delivery of a polynucleotide for therapeutic purposes is commonly called gene therapy. The delivery of a polynucleotide can lead to modification of the genetic material present in the target cell. If the polynucleotide contains an expressible gene, then the expression cassette is subject to the regulatory controls that govern the expression of endogenous genes in the chromosomes. The term transient transfectant refers to a cell which has taken up a polynucleotide but has not integrated the polynucleotide into its genomic DNA.

Intravascular and vessel—The term intravascular refers to an intravascular route of administration that enables a polymer, oligonucleotide, or polynucleotide to be delivered to cells more evenly distributed than direct injections. Intravascular herein means within an internal tubular structure called a vessel that is connected to a tissue or organ within the body of an animal, including mammals. Vessels comprise internal hollow tubular structures connected to a tissue or organ within the body. Bodily fluid flows to or from the body part within the cavity of the tubular structure. Examples of bodily fluid include blood, lymphatic fluid, or bile. Examples of vessels include arteries, arterioles, capillaries, venules, sinusoids, veins, lymphatics, and bile ducts. Afferent blood vessels of organs are defined as vessels which are directed towards the organ or tissue and in which blood flows towards the organ or tissue under normal physiological conditions. Conversely, efferent blood vessels of organs are defined as vessels which are directed away from the organ or tissue and in which blood flows away from the organ or tissue under normal physiological conditions. In the liver, the hepatic vein is an efferent blood vessel since it normally carries blood away from the liver into the inferior vena cava. Also in the liver, the portal vein and hepatic arteries are afferent blood vessels in relation to the liver since they normally carry blood towards the liver. Insertion of the inhibitor or inhibitor complex into a vessel enables the inhibitor to be delivered to parenchymal cells more efficiently and in a more even distribution compared with direct parenchymal injections.

Modification—A molecule is modified, to form a modification through a process called modification, by a second molecule if the two become bonded through a covalent bond. That is, the two molecules form a covalent bond between an atom form one molecule and an atom from the second molecule resulting in the formation of a new single molecule. A chemical covalent bond is an interaction, or bond, between two atoms in which there is a sharing of electron density. Modification also means an interaction between two molecules through a noncovalent bond. For example crown ethers can form noncovalent bonds with certain amine groups.

Salt—A salt is any compound containing ionic bonds; i.e., bonds in which one or more electrons are transferred completely from one atom to another. Salts are ionic compounds that dissociate into cations and anions when dissolved in solution and thus increase the ionic strength of a solution.

Pharmaceutically Acceptable Salt—Pharmaceutically acceptable salt means both acid and base addition salts.

Pharmaceutically Acceptable Acid Addition Salt—A pharmaceutically acceptable acid addition salt is a salt that retains the biological effectiveness and properties of the free base, is not biologically or otherwise undesirable, and is formed with inorganic acids such as hydrochloric acid, hydrobromic acid, sulfuric acid, nitric acid, phosphoric acid and the like, and organic acids such as acetic acid, propionic acid, pyruvic acid, maleic acid, malonic acid, succinic acid, fumaric acid, tartaric acid, citric acid, benzoic acid, mandelic acid, methanesulfonic acid, ethanesulfonic acid, p-toluenesulfonic acid, salicylic acid, trifluoroacetic acid, and the like.

Pharmaceutically Acceptable Base Addition Salt—A pharmaceutically acceptable base addition salt is a salts that retains the biological effectiveness and properties of the free acid, is not biologically or otherwise undesirable, and is prepared from the addition of an inorganic organic base to the free acid. Salts derived from inorganic bases include, but are not limited to, sodium, potassium, calcium, lithium, ammonium, magnesium, zinc, and aluminum salts and the like. Salts derived from organic bases include, but are not limited to, salts of primary secondary, and tertiary amines, such as methylamine, triethylamine, and the like.

Salt Stabilized Complex—A salt stabilized complex is a complex that shows stability when exposed to 150 mM NaCl solution. Stability in this case is indicated by a stable particle size reading (less than a 20% change over 60 min) for the complex in 150 mM NaCl solution. Stability in this case is also indicated by no decondensation of the DNA (less than a 20% change over 60 min) within the complex for the complex in 150 mM NaCl solution.

Interpolyelectrolyte Complexes—An interpolyelectrolyte complex is a noncovalent interaction between polyelectrolytes of opposite charge.

Functional group: Functional groups include cell targeting signals, nuclear localization signals, compounds that enhance release of contents from endosomes or other intracellular vesicles (releasing signals), and other compounds that alter the behavior or interactions of the compound or complex to which they are attached. Additionally, a functional group also means a chemical functional group that can undergo further chemical reactions. Examples include but are not limited to hydroxyl groups, amine groups, thiols, carboxylic acids, aldehydes, and ketones.

Cell targeting signals—Cell targeting signals are any signals that enhance the association of the biologically active compound with a cell. These signals can modify a biologically active compound such as drug or nucleic acid and can direct it to a cell location (such as tissue) or location in a cell (such as the nucleus) either in culture or in a whole organism. The signal may increase binding of the compound to the cell surface and/or its association with an intracellular compartment. By modifying the cellular or tissue location of the foreign gene, the function of the biologically active compound can be enhanced. The cell targeting signal can be, but is not limited to, a protein, peptide, lipid, steroid, sugar, carbohydrate, (non-expressing) polynucleic acid or synthetic compound. Cell targeting signals such as ligands enhance cellular binding to receptors. A variety of ligands have been used to target drugs and genes to cells and to specific cellular receptors. The ligand may seek a target within the cell membrane, on the cell membrane or near a cell. Binding of ligands to receptors typically initiates endocytosis. Ligands include agents that target to the asialoglycoprotein receptor by using asiologlycoproteins or galactose residues. Other proteins such as insulin, EGF, or transferrin can be used for targeting. Peptides that include the RGD sequence can be used to target many cells. Chemical groups that react with thiol, sulfhydryl, or disulfide groups on cells can also be used to target many types of cells. Folate and other vitamins can also be used for targeting. Other targeting groups include molecules that interact with membranes such as lipids, fatty acids, cholesterol, dansyl compounds, and amphotericin derivatives. In addition viral proteins could be used to bind cells.

After interaction of a compound or complex with the cell, other targeting groups can be used to increase the delivery of the biologically active compound to certain parts of the cell.

Nuclear localization signals—Nuclear localizing signals enhance the targeting of the pharmaceutical into proximity of the nucleus and/or its entry into the nucleus during interphase of the cell cycle. Such nuclear transport signals can be a protein or a peptide such as the SV40 large T antigen NLS or the nucleoplasmin NLS. These nuclear localizing signals interact with a variety of nuclear transport factors such as the NLS receptor (karyopherin alpha) which then interacts with karyopherin beta. The nuclear transport proteins themselves could also function as NLS's since they are targeted to the nuclear pore and nucleus. For example, karyopherin beta itself could target the DNA to the nuclear pore complex. Several peptides have been derived from the SV40 T antigen. Other NLS peptides have been derived from the hnRNP A1 protein, nucleoplasmin, c-myc, etc.

Membrane active compounds—Many biologically active compounds, in particular large and/or charged compounds, are incapable of crossing biological membranes. In order for these compounds to enter cells, the cells must either take them up by endocytosis, i.e., into endosomes, or there must be a disruption of the cellular membrane to allow the compound to cross. In the case of endosomal entry, the endosomal membrane must be disrupted to allow for movement out of the endosome and into the cytoplasm. Either entry pathway into the cell requires a disruption or alteration of the cellular membrane. Compounds that disrupt membranes or promote membrane fusion are called membrane active compounds. These membrane active compounds, or releasing signals, enhance release of endocytosed material from intracellular compartments such as endosomes (early and late), lysosomes, phagosomes, vesicle, endoplasmic reticulum, golgi apparatus, trans golgi network (TGN), and sarcoplasmic reticulum. Release includes movement out of an intracellular compartment into the cytoplasm or into an organelle such as the nucleus. Releasing signals include chemicals such as chloroquine, bafilomycin or Brefeldin A1, viral components such as influenza virus hemagglutinin subunit HA-2 peptides and other types of amphipathic peptides. The control of when and where the membrane active compound is active is crucial to effective transport. If the membrane active agent is operative in a certain time and place it would facilitate the transport of the biologically active compound across the biological membrane. If the membrane active compound is too active or active at the wrong time, then no transport occurs or transport is associated with cell rupture and cell death. Nature has evolved various strategies to allow for membrane transport of biologically active compounds including membrane fusion and the use of membrane active compounds whose activity is modulated such that activity assists transport without toxicity. Many lipid-based transport formulations rely on membrane fusion and some membrane active peptides' activities are modulated by pH. In particular, viral coat proteins are often pH-sensitive, inactive at neutral or basic pH and active under the acidic conditions found in the endosome.

Cell penetrating compounds—Cell penetrating compounds, which include cationic import peptides (also called peptide translocation domains, membrane translocation peptides, arginine-rich motifs, cell-penetrating peptides, and peptoid molecular transporters) are typically rich in arginine and lysine residues and are capable of crossing biological membranes. In addition, they are capable of transporting molecules to which they are attached across membranes. Examples include TAT (GRKKRRQRRR; SEQ ID 1), VP22 peptide, and an ANTp peptide (RQIKIWFQNRRMKWKK; SEQ ID 2). Cell penetrating compounds are not strictly peptides. Short, non-peptide polymers that are rich in amines or guanidinium groups are also capable of carrying molecules crossing biological membranes. Like membrane active peptides, cationic import peptides are defined by their activity rather than by strict amino acid sequence requirements.

Interaction Modifiers—An interaction modifier changes the way that a molecule interacts with itself or other molecules relative to molecule containing no interaction modifier. The result of this modification is that self-interactions or interactions with other molecules are either increased or decreased. For example cell targeting signals are interaction modifiers which change the interaction between a molecule and a cell or cellular component. Polyethylene glycol is an interaction modifier that decreases interactions between molecules and themselves and with other molecules.

Linkages—An attachment that provides a covalent bond or spacer between two other groups (chemical moieties). The linkage may be electronically neutral, or may bear a positive or negative charge. The chemical moieties can be hydrophilic or hydrophobic. Preferred spacer groups include, but are not limited to C1-C12 alkyl, C1-C12 alkenyl, C1-C12 alkynyl, C6-C18 aralkyl, C6-C₁₈ aralkenyl, C6-C18 aralkynyl, ester, ether, ketone, alcohol, polyol, amide, amine, polyglycol, polyether, polyamine, thiol, thio ether, thioester, phosphorous containing, and heterocyclic. The linkage may or may not contain one or more labile bonds.

Labile Bond—A labile bond is a covalent bond that is capable of being selectively broken. That is, the labile bond may be broken in the presence of other covalent bonds without the breakage of the other covalent bonds. For example, a disulfide bond is capable of being broken in the presence of thiols without cleavage of other bonds, such as carbon-carbon, carbon-oxygen, carbon-sulfur, carbon-nitrogen bonds, which may also be present in the molecule. Labile also means cleavable.

Labile Linkage—A labile linkage is a chemical compound that contains a labile bond and provides a link or spacer between two other groups. The groups that are linked may be chosen from compounds such as biologically active compounds, membrane active compounds, compounds that inhibit membrane activity, functional reactive groups, monomers, and cell targeting signals. The spacer group may contain chemical moieties chosen from a group that includes alkanes, alkenes, esters, ethers, glycerol, amide, saccharides, polysaccharides, and heteroatoms such as oxygen, sulfur, or nitrogen. The spacer may be electronically neutral, may bear a positive or negative charge, or may bear both positive and negative charges with an overall charge of neutral, positive or negative.

pH-Labile Linkages and Bonds—pH-labile refers to the selective breakage of a covalent bond under acidic conditions (pH<7). That is, the pH-labile bond may be broken under acidic conditions in the presence of other covalent bonds that are not broken.

Amphiphilic and Amphipathic Compounds—Amphipathic, or amphiphilic, compounds have both hydrophilic (water-soluble) and hydrophobic (water-insoluble) parts.

Polymers—A polymer is a molecule built up by repetitive bonding together of smaller units called monomers. In this application the term polymer includes both oligomers which have two to about 80 monomers and polymers having more than 80 monomers. The polymer can be linear, branched network, star, comb, or ladder types of polymer. The polymer can be a homopolymer in which a single monomer is used or can be copolymer in which two or more monomers are used. Monomers themselves may be polymers. Types of copolymers include alternating, random, block and graft.

The main chain of a polymer is composed of the atoms whose bonds are required for propagation of polymer length. The side chain of a polymer is composed of the atoms whose bonds are not required for propagation of polymer length.

Step Polymerization—In step polymerization, the polymerization occurs in a stepwise fashion. Polymer growth occurs by reaction between monomers, oligomers and polymers. No initiator is needed since there is the same reaction throughout and there is no termination step so that the end groups are still reactive. The polymerization rate decreases as the functional groups are consumed.

Typically, step polymerization is done either of two different, ways. One way, the monomer has both reactive functional groups (A and B) in the same molecule so that
A-B yields -[A-B]
   Or the other approach is to have two difunctional monomers.
A-A+B-B yields -[A-A-B-B]
   Yet another approach is to have one difunctional monomer so that
A-A plus another agent yields -[A-A]-.

Chain Polymerization—In chain-reaction polymerization growth of the polymer occurs by successive addition of monomer units to limited number of growing chains. The initiation and propagation mechanisms are different and there is usually a chain-terminating step. The polymerization rate remains constant until the monomer is depleted.

Other Components of the Monomers and Polymers—The polymers have other groups that increase their utility. These groups can be incorporated into monomers prior to polymer formation or attached to the polymer after its formation. These groups include: Targeting Groups—such groups are used for targeting the polymer-nucleic acid complexes to specific cells or tissues. Examples of such targeting agents include agents that target to the asialoglycoprotein receptor by using asialoglycoproteins or galactose residues. Other proteins such as insulin, EGF, or transferrin can be used for targeting. Peptides that include the RGD sequence can be used to target many cells. Chemical groups that react with thiol, sulthydryl, or disulfide groups on cells can also be used to target many types of cells. Folate and other vitamins can also be used for targeting. Other targeting groups include molecules that interact with membranes such as fatty acids, cholesterol, dansyl compounds, and amphotericin derivatives.

A variety of ligands have been used to target drugs and genes to cells and to specific cellular receptors. The ligand may seek a target within the cell membrane, on the cell membrane or near a cell. Binding of ligands to receptors typically initiates endocytosis. Ligands could also be used for DNA delivery that bind to receptors that are not endocytosed. For example peptides containing RGD peptide sequence that bind integrin receptor could be used. In addition viral proteins could be used to bind the complex to cells. Lipids and steroids could be used to directly insert a complex into cellular membranes.

The polymers can also contain cleavable groups within themselves. When attached to the targeting group, cleavage leads to reduce interaction between the complex and the receptor for the targeting group. Cleavable groups include but are not restricted to disulfide bonds, diols, diazo bonds, ester bonds, sulfone bonds, acetals, ketals, enol ethers, enol esters, enamines and imines.

Polyelectrolyte—A polyelectrolyte, or polyion, is a polymer possessing more than one charge, i.e. the polymer contains groups that have either gained or lost one or more electrons. A polycation is a polyelectrolyte possessing net positive charge, for example poly-L-lysine hydrobromide. The polycation can contain monomer units that are charge positive, charge neutral, or charge negative, however, the net charge of the polymer must be positive. A polycation also can mean a non-polymeric molecule that contains two or more positive charges. A polyanion is a polyelectrolyte containing a net negative charge. The polyanion can contain monomer units that are charge negative, charge neutral, or charge positive, however, the net charge on the polymer must be negative. A polyanion can also mean a non-polymeric molecule that contains two or more negative charges. The term polyelectrolyte includes polycation, polyanion, zwitterionic polymers, and neutral polymers. The term zwitterionic refers to the product (salt) of the reaction between an acidic group and a basic group that are part of the same molecule.

Steric Stabilizer—A steric stabilizer is a long chain hydrophilic group that prevents aggregation by sterically hindering particle to particle or polymer to polymer electrostatic interactions. Examples include: alkyl groups, PEG chains, polysaccharides, alkyl amines. Electrostatic interactions are the non-covalent association of two or more substances due to attractive forces between positive and negative charges.

Sterics—Steric hindrance, or sterics, is the prevention or retardation of a chemical reaction because of neighboring groups on the same molecule.

Lipid—Any of a diverse group of organic compounds that are insoluble in water, but soluble in organic solvents such as chloroform and benzene. Lipids contain both hydrophobic and hydrophilic sections. The term lipids is meant to include complex lipids, simple lipids, and synthetic lipids.

Complex Lipids—Complex lipids are the esters of fatty acids and include glycerides (fats and oils), glycolipids, phospholipids, and waxes.

Synthetic Lipids—Synthetic lipids includes amides prepared from fatty acids wherein the carboxylic acid has been converted to the amide, synthetic variants of complex lipids in which one or more oxygen atoms has been substituted by another heteroatom (such as Nitrogen or Sulfur), and derivatives of simple lipids in which additional hydrophilic groups have been chemically attached. Synthetic lipids may contain one or more labile groups.

Glycolipids—Glycolipids are sugar containing lipids. The sugars are typically galactose, glucose or inositol.

Phospholipids—Phospholipids are lipids having both a phosphate group and one or more fatty acids (as esters of the fatty acid). The phosphate group may be bound to one or more additional organic groups.

Fats—Fats are glycerol esters of long-chain carboxylic acids. Hydrolysis of fats yields glycerol and a carboxylic acid—a fatty acid. Fatty acids may be saturated or unsaturated (contain one or more double bonds).

Surfactant—A surfactant is a surface active agent, such as a detergent or a lipid, which is added to a liquid to increase its spreading or wetting properties by reducing its surface tension. A surfactant refers to a compound that contains a polar group (hydrophilic) and a non-polar (hydrophobic) group on the same molecule. A cleavable surfactant is a surfactant in which the polar group may be separated from the nonpolar group by the breakage or cleavage of a chemical bond located between the two groups, or to a surfactant in which the polar or non-polar group or both may be chemically modified such that the detergent properties of the surfactant are destroyed.

Detergent—Detergents are compounds that are soluble in water and cause nonpolar substances to go into solution in water. Detergents have both hydrophobic and hydrophilic groups

Micelle—Micelles are microscopic vesicles that contain amphipathic molecules but do not contain an aqueous volume that is entirely enclosed by a membrane. In micelles the hydrophilic part of the amphipathic compound is on the outside (on the surface of the vesicle). In inverse micelles the hydrophobic part of the amphipathic compound is on the outside. The inverse micelles thus contain a polar core that can solubilize both water and macromolecules within the inverse micelle.

Liposome—Liposomes are microscopic vesicles that contain amphipathic molecules and contain an aqueous volume that is entirely enclosed by a membrane.

Drug Delivery—Drug delivery is the delivery of a biologically active compound. A biologically active compound, such as siRNA, is delivered if it becomes associated with the cell or organism. The compound can be in the circulatory system, intravessel, extracellular, on the membrane of the cell or inside the cytoplasm, nucleus, or other organelle of the cell.

Routes of Administration—Parenteral routes of administration include intravascular (intravenous, intra-arterial), intramuscular, intraparenchymal, intradermal, subdermal, subcutaneous, intratumor, intraperitoneal, intrathecal, subdural, epidural, and intralymphatic injections that use a syringe and a needle or catheter. An intravascular route of administration enables siRNA to be delivered to cells more evenly distributed than direct injections. Intravascular herein means within a tubular structure called a vessel that is connected to a tissue or organ within the body. Within the cavity of the tubular structure, a bodily fluid flows to or from the body part. Examples of bodily fluid include blood, cerebrospinal fluid (CSF), lymphatic fluid, or bile. Examples of vessels include arteries, arterioles, capillaries, venules, sinusoids, veins, lymphatics, and bile ducts. The intravascular route includes delivery through the blood vessels such as an artery or a vein. An administration route involving the mucosal membranes is meant to include nasal, bronchial, inhalation into the lungs, or via the eyes. Other routes of administration include intraparenchymal into tissues such as muscle (intramuscular), liver, brain, and kidney. Transdermal routes of administration have been effected by patches and ionotophoresis. Other epithelial routes include oral, nasal, respiratory, and vaginal routes of administration.

### EXAMPLES

### Example 1

### Polymerization of N,N-Dimethylformamide (MC1015)

Method A: A solution of HCl in diethyl ether (1 mL, 1.0 M, Aldrich Chemical Company) was cooled to −78° C. in a dry ice/acetone bath under N₂. N,N-Dimethylformamide (85 mg, 1.2 mmol, anhydrous, Aldrich Chemical Company) was added dropwise. The resulting precipitate was isolated by centrifugation, washed with diethylether (2×2 mL), dried under a N₂ stream, and placed under high vacuum to afford the imidate (30 mg, 23%). The resulting imidate was dissolved in DMF (300 μL, anhydrous, Aldrich Chemical Company) and allowed to stand at room temperature for 3 days.

Method B: N,N-Dimethylformamide (47.2 g, 0.646 mol, anhydrous, Aldrich Chemical Company) was cooled to −20° C., and HCl gas was bubbled through the solution over 30 min. The resulting solution was warmed to room temperature under a blanket of N₂ to afford a clear viscous solution.

### Example 2

### Polymerization of N,N-Dibutylformamide

N,N-Dibutylformamide (4.3 g, 0.027 mol, Aldrich Chemical Company) was cooled to −20° C., and HCl gas was bubbled through the solution over 30 min. The resulting solution was warmed to room temperature under a blanket of N₂ to afford a clear viscous solution.

### Example 3

### Polymerization of N,N-Dimethylthioformamide

N,N-Dimethylthioformamide (5.2 g, 0.059 mol, Aldrich Chemical Company) was cooled to −20° C., and HCl gas was bubbled through the solution over 30 min. A white crystalline precipitate formed in the solution. As the reaction mixture was warmed to room temperature under a blanket of N₂, the precipitate dissolved into solution to afford a yellow solution.

### Example 4

### Rhodamine-DNA Condensation with polymerized (N,N-dimethylformamide (MC1015)

Rhodamine labeled DNA (RhDNA) was prepared according to the manufacturers protocol (Mirus Bio Corporation) for determination of the level of DNA condensation (Trubetskoy 1999). To H₂O (500 μL), was added RhDNA/DNA (2.5 μL of a 2 μg/μL solution of 1:9 by wt RhDNA/DNA in water), the solution was mixed and the fluorescent intensity was measured on a spectrophotometer (Varian Cary Eclipse Fluorescence Spectrophotometer, Ex=559 nm, Em=576 nm). Polymerized N,N-dimethylformamide (MC1015, 1 μg, 1 μL of 1 μg/mL solution in DMF synthesized using Method A) was added, the solution was vortexed, and the fluorescence intensity was measured on the spectrophotometer. Subsequent additions of MC1015 were added to the RhDNA solution until no further decrease in fluorescence intensity was observed.

Results: Fluorescence Quenching Indicates a Condensed pDNA Particle.

| Complex | Fluorescence Intensity (AU) |
|---|---|
| 5 μg RhDNA/DNA (1 μg/9 μg) | 273.159 |
| +1 μg MC1015 | 354.889 |
| +1 μg MC1015 | 261.723 |
| +1 μg MC1015 | 108.184 |
| +1 μg MC1015 | 50.205 |

The results indicate that 5 μg of DNA was fully condensed with 4 μg of MC1015.

### Example 5

### Mouse Tail Vein Injections of pDNA (pCI Luc)/MC1015 Complexes For Luciferase Expression

MC510 was prepared as follows: To a solution of poly(methyl vinyl ether-alt-maleic anhydride) (50 mg, Aldrich Chemical Company) in 10 mL of anhydrous tetrahydrofuran was added 100 mg of histamine. The resulting solution was stirred for 1 hour followed by the addition of 10 mL water. The solution was stirred for another hour and then placed into a 12,000 MW cutoff dialysis tubing and dialyzed against 7×4 L water over a one week period, and then concentrated to 1 mL volume by lyophilization.

Three complexes for injection were prepared as follows:
Complex I: pDNA (pCI Luc, 20 μg) mixed with MC1015 (60 μg) in 150 mM NaCl (5.0 mL).
Complex II: pDNA (pCI Luc, 20 μg) mixed with MC1015 (60 μg) in 150 mM NaCl (5.0 mL). To the resulting complex was added poly-L-lysine, succinylated (200 μg, Sigma Chemical), and the resulting complex was vortexed to mix.
Complex III: pDNA (pCI Luc, 20 μg) mixed with MC1015 (60 μg) in 150 mM NaCl (5.0 mL). To the resulting solution was added MC510 (200 μg), and the resulting complex was vortexed to mix.

Solutions (2.5 mL) were injected into the tail vein of ICR mice (n=4) using a 30 gauge, 0.5 inch needle, manually over 1-3 sec (Zhang et al. 2004). Luciferase expression was determined 24 hrs post injection as previously reported (Wolff et al 19990). A Lumat LB 9507 (EG&G Berthold, Bad-Wildbad, Germany) luminometer was used.

Results: 2.5 mL Injections RLU

| Complex | Liver 100x | | | | |
|---|---|---|---|---|---|
| Number | dilution | Spleen | Lung | Heart | Kidney |
| Complex I | 967,814 | 416,644 | 158,241 | 36,698 | 117,014 |
| Complex II | 3,713,5931 | 187,317 | 283,996 | 80,694 | 167,899 |
| Complex III | 2,114,335 | 227,932 | 106,590 | 38,805 | 181,077 |

These results indicate that the pDNA is being released from the pDNA/MC1015 complexes, and is accessible for transcription.

### Example 6

### Mouse Tail Vein Injections of pDNAs (pCI Luc and pCI Renilla) with siRNA (GL3-153 or EGFP-64) for mRNA knockout

GL3-153 siRNA is an annealed ds siRNA against Luc with a sequence of 2′OH-CUU ACG CUG AGU ACU UCG AdTdT (SEQ ID 3) and its compliment 2′OH-UCG AAG UAC UCA GCG UAA GdTdT (SEQ ID 4; GL-3, TriLink BioTechnologies Inc.)

EGFP-64 siRNA is an annealed ds siRNA against EGFP with the sequence of 5′ GAC GUA AAC GGC CAC AAG UGC 3′ (SEQ ID 5) and it's compliment 3′CG CUG CAU UUG CCG GUG UUC A 5′ (SEQ ID 6; Dharmacon).

Seven complexes were prepared as follows:
Complex I: pDNA's (pCI Luc, 30 μg and pCI Renilla, 3 μg) in Ringers (7.3 mL) was mixed with 150 mM NaCl (0.2 mL).
Complex II: GL3-153 (15 μg) in 150 mM NaCl (0.2 mL) was added to pDNA's (pCI Luc, 30 μg and pCI Renilla, 3 μg) in Ringers (7.3 mL) and the solution was mixed.
Complex III: EGFP-64 (15 μg) in 150 mM NaCl (0.2 mL) was added to pDNA's (pCI Luc, 30 μg and pCI Renilla, 3 μg) in Ringers (7.3 mL) and the solution was mixed.
Complex IV: GL3-153 (15 μg) was mixed with MC1015 (24 μg) in 150 mM NaCl (0.2 mL) and MC510 (15 μg) was added and mixed. This complex was added to pDNA's (pCI Luc, 30 μg and pCI Renilla, 3 μg) in Ringers (7.3 mL) and the solution was mixed.
Complex V: EGFP-64 (15 μg) was mixed with MC1015 (24 μg) in 150 mM NaCl (0.2 mL) and MC510 (150 μg) was added and mixed. This complex was added to pDNA's (pCI Luc, 30 μg and pCI Renilla, 3 μg) in Ringers (7.3 mL) and the solution was mixed.
Complex VI: GL3-153 (15 μg) was mixed with MC1015 (24 μg) in 150 mM NaCl (0.2 mL) and succinylated poly-L-lysine (150 μg, Sigma Chemical) was added and mixed. The complex was added to pDNA's (pCI Luc, 30 μg and pCI Renilla, 3 μg) in Ringers (7.3 mL) and the solution was mixed.
Complex VII: EGFP-64 (15 μg) was mixed with MC1015 (24 μg) in 150 mM NaCl (0.2 mL) and succinylated poly-L-lysine (150 μg, Sigma Chemical) was added and mixed. The complex was added to pDNA's (pCI Luc, 30 μg and pCI Renilla, 3 μg) in Ringers (7.3 mL) and the solution was mixed.

Solutions (2.5 mL) were injected into the tail vein of ICR mice (n=4) using a 30 gauge, 0.5 inch needle, manually over 1-3 sec (Zhang et al. 2004). Luciferase expression was determined 24 hrs post injection as previously reported (Wolff et al. 1990). A Lumat LB 9507 (EG&G Berthold, Bad-Wildbad, Germany) luminometer was used.

Results indicate a decreased level of pCI Luc/pCI Renilla DNA expression in complexes IV and VI over pCI Luc/pCI Renilla (Complex I). These results also indicate that the GL3-153 is being released from the complex, and is accessible.

Results: 2.5 mL Injections RLU 10× Dilution

| Complex Number | Luc | Renilla | Luc/Ren |
|---|---|---|---|
| Complex I: | 33,527,330 | 31,631,340 | 106 |
| Complex I: | 93,366,860 | 59,445,510 | 157 |
| Complex II: | 8,833,060 | 65,054,070 | 14 |
| Complex II: | 2,399,930 | 10,353,310 | 23 |
| Complex III: | 198,397,490 | 243,643,090 | 81 |
| Complex III: | 30,235,340 | 18,591,320 | 163 |
| Complex IV: | 959,670 | 3,985,390 | 24 |
| Complex IV: | 18,242,640 | 98,315,430 | 19 |
| Complex V: | 78,816,880 | 116,056,860 | 68 |
| Complex V: | 6,069,120 | 6,355,580 | 95 |
| Complex VI: | 27,808,080 | 144,055,900 | 19 |
| Complex VI: | 14,234,590 | 121,553,050 | 12 |
| Complex VII: | 80,143,540 | 136,728,650 | 59 |
| Complex VII: | 83,054,470 | 117,951,620 | 70 |

### Example 7

### Mouse Portal Vein Injections of pDNA/MC1015 Complexes

Four complexes were prepared as follows:
Complex I: pDNA (pCI Luc 60 μg) was mixed with MC1015 (36 μg) in 0.9% NaCl (600 μL).
Complex II: pDNA (pCI Luc 60 μg) was mixed with MC1015 (36 μg) in 0.9% NaCl (600 μL). MC576 (24 μg, C₁₂H₂₅OSi(CH₃)₂—CH₂CH₂—Si(CH₃)₂Cl) was added and the solution was mixed.
Complex III: pDNA (pCI Luc 60 μg) was mixed with MC1015 (36 μg) in 0.9% NaCl (600 μL). MC510 (24 μg) was added and the solution was mixed.
Complex IV: pDNA (pCI Luc 60 μg) was mixed with MC1015 (36 μg) in 5 mM MES pH 5.0, isotonic glucose (600 μL). MC510 (24 μg) was added and the solution was mixed.
Complex V: pDNA (pCI Luc 60 μg) in 0.9% NaCl (600 μL).

Solutions were injected into the portal vein of ICR mice. The livers were exposed through a ventral midline incision, and the complexes were injected using a Harvard Apparatus PH 2000 programmable pump programmed to deliver 200 μL over 4 seconds into the portal vein using a 30-gauge, ½-inch needle and 1-ml syringe. Several 5×1 mm, Kleinert-Kutz microvessel clips (Edward Weck, Inc., Research Triangle Park, N.C.) were applied prior to and for 2 minutes following the injection in order to clamp the IVC (lower), SVC (upper), and the portal vein. Anesthesia was obtained from inhalation of isoflurane (Abbott Laboratories) as needed. One day after injection, the animals were sacrificed, and luciferase expression was assayed from the liver.

Results: Luciferase Expression RLU for Livers

| Complex Number | n1 | n2 |
|---|---|---|
| Complex I | 2,937 | 87,368 |
| Complex II | 24,699 | 120,960 |
| Complex III | 112,029 | 8,398 |
| Complex IV | 12,552 | 17,152 |
| Complex V | 2,694 | |

The results indicate that MC1105 improves expression of the pDNA in the liver following delivery to the portal vein.

### Example 8

### Mouse Bile Duct Injections

Six complexes were made as follows:
Complex I: pDNA (pCI Luc 60 μg) was mixed with MC1015 (60 μg) in 5 mM MES pH 5.0, isotonic glucose (600 μL).
Complex II: pDNA (pCI Luc 60 μg) was mixed with MC1015 (192 μg) in 5 mM MES pH 5.0, isotonic glucose (600 μL).
Complex III: pDNA (pCI Luc 60 μg) was mixed with MC1015 (144 μg) in 5 mM MES pH 5.0, isotonic glucose (600 mL).
Complex IV: pDNA (pCI Luc 60 μg) was mixed with MC1015 (192 μg) in 5 mM MES pH 5.0, isotonic glucose (600 μL). MC899 (15 μg) was added and the solution mixed.
Complex V: pDNA (pCI Luc 60 μg) was mixed with MC1015 (192 μg) in 5 mM MES pH 5.0, isotonic glucose (600 μL). MC899 (30 μg) was added and the solution mixed.
Complex VI: pDNA (pCI Luc 60 μg) was mixed with MC1015 (192 μg) in 5 mM MES pH 5.0, isotonic glucose (600 μL). MC899 (60 μg) was added and the solution mixed.

Bile duct injections on ICR mice (n=2) were performed using a Harvard Apparatus PH 2000 programmable pump with a 30-gauge, 1/2 inch needle and 1 ml syringe. The pump was programmed to deliver 200 μL over 4 seconds. A 5×1 mm, Kleinert Kutz microvessel clip was used to occlude the bile duct downstream from the point of injection in order to prevent flow to the duodenum and away from the liver. The gallbladder inlet was not occluded. In these injections, the junction of the hepatic vein and caudal vena cava were not clamped. Additionally, the portal vein and hepatic artery were not clamed for the injection. MC899 is the copolymer resulting from the EDC coupling of 3-aminopropyl imidazole with the poly(acrylic acid -co-maleic acid) sodium salt (Aldrich Chemical Company, 3 eq acid, 1.25 eq imidazole).

Results: Luciferase Expression (RLU) for Livers 10× Diluted

| Complex Number | n1 | n2 |
|---|---|---|
| Complex I | 31,560 | 154,121 |
| Complex II | 35,088 | 103,056 |
| Complex III | 52,944 | 155,637 |
| Complex IV | 125,382 | 105,063 |
| Complex V | 96,724 | 461,206 |
| Complex VI | 46,403 | 14,159 |

The results indicate that the described binary and ternary complexes are able to deliver pDNA to the liver via the bile duct.

### Example 9

### Mouse Low Pressure Bile Duct Injections—Dual Luciferase Assay

Four complexes were made as follows:
Complex I: pDNA (pCI Luc 72 μg, pCI Renilla 8 μg) with GL3-153 (80 μg) and MC1015 (128 μg) was mixed in 5 mM MES pH 5.0, isotonic glucose (800 μL).
Complex II: pDNA (pCI Luc 72 μg, pCI Renilla 8 μg) with EGP-64 (80 μg) and MC1015 (128 μg) was mixed in 5 mM MES pH 5.0, isotonic glucose (800 μL).
Complex III: pDNA (pCI Luc 72 μg, pCI Renilla 8 μg) with GL3-153 (80 μg), MC1015 (128 μg) and MC576 (40 μg) was mixed in 5 mM MES pH 5.0, isotonic glucose (800 μL).
Complex IV: pDNA (pCI Luc 72 μg, pCI Renilla 8 μg) with EGP-64 (80 μg), MC1015 (128 μg) and MC576 (40 kg) was mixed in 5 mM MES pH 5.0, isotonic glucose (800 μL).

Bile duct injections on ICR mice (n=3) were performed using a Harvard Apparatus PH 2000 programmable pump with a 30-gauge, 1/2 inch needle and 1 ml syringe. The pump was programmed to deliver 200 μL over 4 seconds. A 5×1 mm, Kleinert Kutz microvessel clip was used to occlude the bile duct downstream from the point of injection in order to prevent flow to the duodenum and away from the liver. The gallbladder inlet was not occluded. In these injections, the junction of the hepatic vein and caudal vena cava were not clamped. Additionally, the portal vein and hepatic artery were not clamed for the injection.

| Complex Number | Luc | Renilla | Luc/Ren |
|---|---|---|---|
| Complex I | 104,120 | 1,022,900 | 10 |
| Complex I | 132,890 | 1,327,810 | 10 |
| Complex I | 283,900 | 1,921,600 | 15 |
| Complex II | 444,710 | 756,390 | 59 |
| Complex II | 1,923,740 | 2,662,680 | 72 |
| Complex II | 2,629,070 | 2,978,080 | 88 |
| Complex III | 129,250 | 262,310 | 49 |
| Complex III | 272,450 | 320,940 | 85 |
| Complex III | 186,220 | 995,150 | 19 |
| Complex IV | 490,580 | 692,710 | 71 |
| Complex IV | 2,003,830 | 2,210,370 | 91 |
| Complex IV | 1,521,120 | 1,799,400 | 84 |

The results indicate a decrease in luciferase expression for Complex I and Complex III indicating knock out.

### Example 10

### Polymerization of a mixture of N,N-Dimethylformamide and N,N-Dibutylformamide

### A. MC1049: 2.5% N,N-Dibutylformamide in N,N-Dimethylformamide

N,N-Dibutylformamide (0.06 mL, 0.32 mmol, Aldrich Chemical Company) was added to a solution of N,N-Dimethylformamide (1 mL, 13 mmol, Aldrich Chemical Company) and the solution was mixed with vortexing. The solution of mixed formamides was added dropwise to a cooled solution (0° C.) of HCl/Diethylether (15 mL, 1N, Aldrich Chemical Company), resulting in the formation of an orange oil. The reaction mixture was concentrated under a stream of N₂. The resulting oil was washed with hexane (2×10 mL) and dried under a stream of N₂. The oil was placed under high vacuum for 10 min, weighed (1.26 g) and brought up in DMF (12.6 mL, 100 mg/mL). The resulting solution was stirred at room temperature overnight.

### B. MC1050: 5% N,N-Dibutylformamide/N,N-Dimethylformamide

N,N-Dibutylformamide (0.12 mL, 0.65 mmol, Aldrich Chemical Company) was added to a solution of N,N-Dimethylformamide (1 mL, 13 mmol, Aldrich Chemical Company) and mixed with vortexing. The solution of mixed formamides was added dropwise to a cooled solution (0° C.) of HCl/Diethylether (15 mL, 1N, Aldrich Chemical Company), resulting in the formation of an orange oil. The reaction mixture was concentrated under a stream of N₂. The resulting oil was washed with hexane (2×10 mL) and dried under a stream of N₂. The oil was placed under high vacuum for 10 min, weighed (0.793 g) and brought up in DMF (7.9 mL, 100 mg/mL). The resulting solution was stirred at room temperature overnight.

### MC1051: 10% N,N-Dibutylformamide/N,N-Dimethylformamide

N,N-Dibutylformamide (0.23 mL, 1.3 mmol, Aldrich Chemical Company) was added to a solution of N,N-Dimethylformamide (1 mL, 13 mmol, Aldrich Chemical Company) and mixed with vortexing. The solution of mixed formamides was added dropwise to a cooled solution (0° C.) of HCl/Diethylether (15 mL, 1N, Aldrich Chemical Company), resulting in the formation of an orange oil. The reaction mixture was concentrated under a stream of N₂. The resulting oil was washed with hexane (2×10 mL) and dried under a stream of N₂. The oil was placed under high vacuum for 10 min, weighed (1.30 g) and brought up in DMF (13 mL, 100 mg/mL). The resulting solution was stirred at room temperature overnight.

### Example 11

### Rh-DNA Condensation with Mixed Formamide Derived Copolymers

Rhodamine labeled DNA (RhDNA) was prepared according to the manufacturers protocol (Mirus Bio Corporation) for determination of the level of DNA condensation (Trubetskoy 1999). To H₂O (500 μL), was added RhDNA (μL of a 1 μg/μL solution in water), the solution was mixed and the fluorescent intensity was measured on a spectrophotometer (Varian Cary Eclipse Fluorescence Spectrophotometer, Ex=559 nm, Em=576 nm). Copolymers from example 10 (MC1049, MC1050, MC1051, all 1 mL of 2 μg/μL solution in DMF) were added, the solutions were mixed, and the fluorescence intensity was measured on the spectrophotometer. Subsequent additions of the copolymers were added to the RhDNA solution until no further decrease in fluorescence intensity was observed.

Results: Fluorescence Quenching Indicates a Condensed pDNA Particle.

| Complex | Fluorescence Intensity (AU) |
|---|---|
| 5 μg RhDNA | 995.916 |
| +2 μg MC1049 | 672.389 |
| +2 μg MC1049 | 410.672 |
| +2 μg MC1049 | 201.150 |
| +2 μg MC1049 | 85.299 |
| +2 μg MC1049 | 47.759 |
| 5 μg RhDNA | 995.916 |
| +2 μg MC1050 | 801.681 |
| +2 μg MC1050 | 548.508 |
| +2 μg MC1050 | 321.639 |
| +2 μg MC1050 | 147.226 |
| +2 μg MC1050 | 71.303 |
| +2 μg MC1050 | 44.261 |
| 5 μg RhDNA | 995.916 |
| +2 μg MC1051 | 794.164 |
| +2 μg MC1051 | 537.948 |
| +2 μg MC1051 | 320.677 |
| +2 μg MC1051 | 170.529 |
| +2 μg MC1051 | 84.078 |
| +2 μg MC1051 | 55.105 |
| +2 μg MC1051 | 45.712 |

The results indicate that the copolymers obtained from the polymerization of formamide mixtures condense RhDNA.

### Example 12

### Rh-DNA Condensation with Mixed Formamide Derived Copolymers and Release of Rh-DNA with Increased pH

To H₂O (500 μL), was added RhDNA (5 μL of a 1 μg/μL solution in water), the solution was mixed and the fluorescent intensity was measured on a spectrophotometer (Varian Cary Eclipse Fluorescence Spectrophotometer, Ex=559 nm, Em=576 nm). Copolymers from example 10 (MC1049, MC1050, MC1051, all 1.5 μL of 10 μg/μL solution in DMF) were added, the solutions were mixed, and the fluorescence intensity was measured on the spectrophotometer. The resulting solution pH was 4. The pH was increased by the addition of NaOH (5 μL of 0.01 N) until full decondensation was achieved as seen by increased fluorescence. Samples were reread after 30 min.

Results:

| Complex | Fluorescence Intensity (AU) |
|---|---|
| Complex I | |
| 5 μg RhDNA | 907.347 |
| +15 μg MC1049 (pH 4) | 59.075 |
| +5 μL 0.01 N NaOH (pH 4.3) | 57.821 |
| +5 μL 0.01 N NaOH (pH 4.7) | 55.504 |
| +5 μL 0.01 N NaOH (pH 5) | 428.046 |
| +10 μL 0.01 N NaOH (pH 5.5) | 869.554 |
| 30 min (pH 5) | 905.613 |
| Complex II | |
| 5 μg RhDNA | 879.632 |
| +15 μg MC1050 (pH 4) | 74.648 |
| +5 μL 0.01 N NaOH (pH 4.3) | 78.407 |
| +5 μL 0.01 N NaOH (pH 4.6) | 89.109 |
| +5 μL 0.01 N NaOH (pH 5) | 835.374 |
| 30 min (pH 5) | 838.765 |
| Complex III | |
| 5 μg RhDNA | 822.768 |
| +15 μg MC1051 (pH 4) | 78.524 |
| +5 μL 0.01 N NaOH (pH 4.3) | 85.461 |
| +5 μL 0.01 N NaOH (pH 4.7) | 96.345 |
| +5 μL 0.01 N NaOH (pH 5) | 909.287 |
| 30 min (pH 5) | 895.262 |
| Complex IV | |
| 5 μg RhDNA | 798.050 |
| +15 μg MC1015 (pH 4) | 87.003 |
| +5 μL 0.01 N NaOH (pH 4.4) | 98.404 |
| +5 μL 0.01 N NaOH (pH 4.7) | 247.010 |
| +5 μL 0.01 N NaOH (pH 5) | 857.795 |
| 30 min (pH 5) | 904.674 |

The results indicate release of the Rh-DNA by the polymer as the pH of the solution is increased to around pH5.

### Example 13

### Mouse Tail Vein Injections of pDNA (pCI Luc)/Polymerized Formamide Complexes for Luciferase Expression

Ten complexes were prepared as follows:
Complex I: pDNA (pCI Luc, 30 μg, 15 μL of 2 μg/μL solution) in 7.5 mL Ringer's solution.
Complex II: pDNA (pCI Luc, 30 μg, 15 μL of 2 μg/μL solution) mixed with MC1049 (90 μg, 9 μL of 10 μg/μL DMF) in 150 mM NaCl (600 μL).
Complex III: pDNA (pCI Luc, 30 μg, 15 μL of 2 μg/μL solution) mixed with MC1050 (90 μg, 9 μL of 10 μg/μL DMF) in 150 mM NaCl (600 μL).
Complex IV: pDNA (pCI Luc, 30 μg, 15 μL of 2 μg/μL solution) mixed with MC1051 (90 μg, 9 μL of 10 μg/μL DMF) in 150 mM NaCl (600 μL).
Complex V: pDNA (pCI Luc, 30 μg, 15 μL of 2 μg/μL solution) mixed with MC1049 (90 μg, 9 μL of 10 μg/μL DMF) in 150 mM NaCl (600 μL). Allowed the complex to sit at room temperature for 5 min. Dar 65C (75 μg, 7.5 μL of 10 μg/μL) was added to the solution and vortexed.
Complex VI: pDNA (pCI Luc, 30 μg, 15 μL of 2 μg/μL solution) mixed with MC1050 (90 μg, 9 μL of 10 μg/μL DMF) in 150 mM NaCl (600 μL). Allowed the complex to sit at room temperature for 5 min. Dar 65C (75 μg, 7.5 μL of 10 μg/μL) was added to the solution and vortexed.
Complex VII: pDNA (pCI Luc, 30 μg, 15 μL of 2 μg/μL solution) mixed with MC1051 (90 μg, 9 mL of 10 μg/μL DMF) in 150 mM NaCl (600 μL). Allowed the complex to sit at room temperature for 5 min. Dar 65C (75 μg, 7.5 μL of 10 μg/μL) was added to the solution and vortexed.
Complex VIII: pDNA (pCI Luc, 30 μg, 15 mL of 2 μg/μL solution) mixed with MC1049 (90 μg, 9 μL of 10 μg/μL DMF) in 150 mM NaCl (600 μL). Allowed the complex to sit at room temperature for 5 min. Dar 65D (75 μg, 7.5 μL of 10 μg/μL) was added to the solution and vortexed.
Complex IX: pDNA (pCI Luc, 30 μg, 15 μL of 2 μg/μL solution) mixed with MCI 050 (90 μg, 9 μL of 10 μg/μL DMF) in 150 mM NaCl (600 μL). Allowed the complex to sit at room temperature for 5 min. Dar 65D (75 μg, 7.5 μL of 10 μg/μL) was added to the solution and vortexed.
Complex X: pDNA (pCI Luc, 30 μg, 15 μL of 2 μg/μL solution) mixed with MC1051 (90 μg, 9 μL of 10 μg/μL DMF) in 150 mM NaCl (600 μL). Allowed the complex to sit at room temperature for 5 min. Dar 65D (75 μg, 7.5 μL of 10 μg/μL) was added to the solution and vortexed.

All complexes were brought up in Ringer's solution (1×, 7.5 mL total volume) with vortexing prior to injection. Solutions (2.5 mL) were injected into the tail vein of ICR mice (n=2) using a 30 gauge, 0.5 inch needle, manually over 1-3 sec (Zhang et al 2004). Luciferase expression was determined 24 hrs post injection as previously reported (Wolff et al 1990). A Lumat LB 9507 (EG&G Berthold, Bad-Wildbad, Germany) luminometer was used. Dar 65C and Dar 65D are the polymers resulting from the polymerization of maleic anhydride and butyl vinylether at 50° C. in toluene (initiated with 1% AIBN for 65C, and 0.5% AIBN for 65D). The polymers were precipitated in pet ether, dried under vacuum, and treated with N-butanol to complete a ring opening of the anhydride.

Results: 2.5 mL Injections (RLU) Livers 100× Dilution

| Complex | n1 | n2 |
|---|---|---|
| Complex I | 4,948,254 | 2,180,999 |
| Complex II | 4,545,001 | 6,309,614 |
| Complex III | 19,430,966 (10x dil) | 18,443,054 (10x dil) |
| Complex IV | 3,805,550 | 877,097 |
| Complex V | 4,740,210 | 293,292 |
| Complex VI | 4,520,404 | 2,984,454 |
| Complex VII | 5,292,890 | 5,781,287 |
| Complex VIII | 5,094,640 | 3,199,345 |
| Complex IX | 4,634,259 | 7,229,992 |
| Complex X | 5,017,524 | 9,587,127 |

The results indicate that the described binary and ternary complexes are able to deliver pDNA to the liver via the tail vein.

### Example 14

### Mouse Portal Vein Injections (200 μL/Injection)

Six complexes were prepared as follows:
Complex I: pDNA (pCI Luc 60 μg, 30 μL of 2 μg/μL solution) was mixed with MC1015 (180 μg, 18 μL of 10 μg/μL DMF) in 0.9% NaCl (600 μL).
Complex II: pDNA (pCI Luc 60 μg, 30 μL of 2 μg/μL solution) was mixed with MC1049 (180 μg, 18 mL of 10 μg/μL DMF) in 0.9% NaCl (600 μL).
Complex III: pDNA (pCI Luc 60 kg, 30 μL of 2 μg/μL solution) was mixed with MC1050 (180 μg, 18 μL of 10 μg/μL DMF) in 0.9% NaCl (600 μL).
Complex IV: pDNA (pCI Luc 60 μg, 30 μL of 2 μg/μL solution) was mixed with MC1050 (360 μg, 36 μL of 10 μg/μL DMF) in 0.9% NaCl (600 λL).
Complex V: pDNA (pCI Luc 60 μg, 30 μL of 2 μg/μL solution) was mixed with MC1051 (180 μg, 18 μL of 10 μg/μL DMF) in 0.9% NaCl (600 μL).
Complex VI: pDNA (pCI Luc 60 μg, 30 μL of 2 μg/μL solution) was mixed with MC1051 (360 μg, 36 μL of 10 μg/μL DMF) in 0.9% NaCl (600 μL).

Solutions were injected into the portal vein of ICR mice. The livers were exposed through a ventral midline incision, and the complexes were injected using a Harvard Apparatus PH 2000 programmable pump programmed to deliver 200 μL over 30 seconds into the portal vein using a 30-gauge, ½-inch needle and 1-ml syringe. Several 5×1 mm, Kleinert-Kutz microvessel clips (Edward Weck, Inc., Research Triangle Park, N.C.) were applied prior to and for 2 minutes following the injection in order to clamp the IVC (lower), SVC (upper), and the portal vein. Anesthesia was obtained from inhalation of isoflurane (Abbott Laboratories) as needed. One day after injection, the animals were sacrificed, and luciferase expression was assayed from the liver.

Results: Luciferase Expression (RLU) for Livers

| Complex Number | n1 | n2 |
|---|---|---|
| Complex I | 9,603 | 41,877 |
| Complex II | 5,386 | 74,368 |
| Complex III | 129,298 | 2,692 |
| Complex IV | 6,634 | 66,484 |
| Complex V | 32,593 | 30,776 |
| Complex VI | 10,327 | 10,133 |

The results indicate that MC1015, MC1049, MC1050, and MC1051 all are able to deliver pDNA in the liver following injection into the portal vein.

### Example 15

### Mouse Saphenous Vein Injections of pDNA (pCI Luc)/Polymerized Formamide Complexes for Luciferase Expression

Five complexes were prepared as follows:
Complex I: pDNA (pCI Luc, 125 μg, 62.5 μL of 2 μg/μL solution) in 150 mM NaCl (3.75 mL).
Complex II: pDNA (pCI Luc, 125 μg, 62.5 μL of 2 μg/μL solution) mixed with MC1015 (375 μg, 37.5 μL of 10 μg/μL DMF) in 150 mM NaCl (3.75 mL).
Complex III: pDNA (pCI Luc, 125 μg, 62.5 μL of 2 μg/μL solution) mixed with MC1049 (375 μg, 37.5 μL of 10 μg/μL DMF) in 150 mM NaCl (3.75 mL).
Complex IV: pDNA (pCI Luc, 125 μg, 62.5 μL of 2 μg/μL solution) mixed with MC1050 (375 μg, 37.5 μL of 10 μg/μL DMF) in 150 mM NaCl (3.75 mL).
Complex V: pDNA (pCI Luc, 125 μg, 62.5 μL of 2 μg/μL solution) mixed with MC1051 (375 μg, 37.5 μL of 10 μg/μL DMF) in 150 mM NaCl (3.75 mL).

Ten mice were injected with a plasmid encoding the-firefly luciferase gene. For each injection, a solution containing the plasmid was inserted into lumen of the saphenous vein animals as follows: A latex tourniquet was wrapped around the upper hind limb just above the quadriceps and tightened into place with a hemostat to block blood flow to and from the leg. A small incision was made to expose the distal portion of the great (or medial) saphenous vein. A 30 gauge needle catheter was inserted into the distal vein and advanced so that the tip of the needle was positioned just above the knee in an antegrade orientation. A syringe pump was used to inject an efflux enhancer solution (0.017% papaverine in 0.25 ml saline) at a flow rate of 4.5 ml/min followed 1-5 min later by injection of 1.5 ml saline containing 50 μg pDNA at a flow rate of 4.5 ml/min. The solution was injected in the direction of normal blood flow through the vein. Two minutes after injection, the tourniquet was removed and bleeding was controlled with pressure and a hemostatic sponge. The incision was closed with 4-0 Vicryl suture. The procedure was completed in ˜10 min. Mice were euthanized at 5 days post-injection and limb muscles were harvested and separated into 6 groups (quadriceps, biceps, hamstring, gastrocnemius, shin and foot). The luciferase activity from each muscle group was determined as previously described (Zhang et al. 2001) and total level of luciferase expression per gram of muscle tissue was determined as previously reported (Wolff et al. 1990). A Lumat LB 9507 (EG&G Berthold, Bad-Wildbad, Germany) luminometer was used.

Results: Luciferase Expression (RLU)

| Complex | | | | | |
|---|---|---|---|---|---|
| Number | Quadriceps | Bicep | Hamstring | Gastrocnemius | Shin |
| Complex I | 13,486,467 | 12,441,155 | 24,835,728 | 16,415,815 | 4,213,843 |
| Complex I | 33,249 | 6,888 | 17,974 | 5,222 | 5,062 |
| Complex II | 2,103,311 | 1,595,217 | 2,065,908 | 3,117,428 | 29,525 |
| Complex II | 5,555,626 | 5,148,498 | 929,501 | 2,684,483 | 46,463 |
| Complex III | 11,980,521 | 18,780,208 | 9,333,930 | 6,435,939 | 690,360 |
| Complex III | 4,160 | 15,403 | 1,389 | 1,123 | 1,507 |
| Complex IV | 16,413,380 | 16,291,077 | 18,948,662 | 22,491,516 | 2,070,813 |
| Complex IV | 33,673 | 23,597 | 5,932 | 2,789 | N/A |
| Complex V | 7,129,377 | 8,322,334 | 7,845,633 | 8,597,729 | 1,145,689 |
| Complex V | 91,880 | 5,225 | 197,965 | 1,655 | N/A |

### Example 16

### In Vitro siRNA Induced Knockdown in 3T3-LUC Cells

Samples were formulated as follows:
Sample 1: OPTI (100 μL)
Sample 2: H₂O (100 μL)+GL2 (1 μL, 100 ng, 0.0075 pmol)
Sample 3: H₂O (100 μL)+GL2 (1 μL, 100 ng, 0.0075 pmol)+TransIT-TKO (TKO)
Sample 4, 5, 6: H₂O (100 μL)+GL2 (1 μL, 100 ng, 0.0075 pmol)+MC1015

NaCl (6 μL of 5 M for 150 mM final concentration) was added to samples 2 through 6 and mixed with vortexing.

Transfection of 3T3-Luc Cells. Samples were prepared as above. GL-2 is an annealed ds siRNA with the sequence 2′OH-CGU ACG CGG AAU ACU UCG AdTdT (SEQ ID 7) and its compliment 2′OH-UCG AAG UAU UCC GCG UAC GdTdT (SEQ ID 8), active against Luc (TriLink BioTechnologies Inc.). Transfections were conducted in duplicate in 12 well plates by covering the cells with 500 μL DMEM with 10% serum and adding 100 μL of transfection sample. Cells were harvested 24 hr post transfection, and read on a luminometer. RLUs are the average of the two wells.

Results: siRNA (12.5 nM)

| | | | % | % |
|---|---|---|---|---|
| # | Sample | Mean RLU | Expression | Confluency |
| 1 | OPTI | 1,779,730 | 100 | 100 |
| 2 | GL2 | 1,124,782 | 63 | 100 |
| 3 | GL2 + TKO (2 uL) | 1,480,606 | 83 | 100 |
| 4 | GL2 + MC1015 (20 ng) | 649,928 | 37 | 100 |
| 5 | GL2 + MC1015 (40 ng) | 395,569 | 22 | 100 |
| 6 | GL2 + MC1015 (80 ng) | 815,813 | 46 | 100 |

The results indicate increased knockout in with binary complexes of MC1015.

### Example 17

### Rh-DNA Condensation with polymerized N,N-dimethylformamide, and decondensation with NaOH

To H₂O (500 μL), was added RhDNA (5 μL of a 2 μg/L solution in water), the solution was mixed and the fluorescent intensity was measured on a spectrophotometer (Varian Cary Eclipse Fluorescence Spectrophotometer, Ex=559 nm, Em=576 nm). MC1015 (10 μg, 5 μL of 2 μg/μL DMF) was added, the solution was mixed, and the fluorescence intensity was measured on the spectrophotometer. The resulting solution pH was 5. The pH was increased by the addition of NaOH (1 μL of 0.1 N) until full decondensation was achieved as seen by increased fluorescence. The sample was again reread after 3 hours.

| Complex | Fluorescence Intensity (AU) |
|---|---|
| 5 μg RhDNA | 741.450 |
| +10 μg MC1015 | 90.547 |
| +1 μL 0.1 N NaOH | 56.815 |
| +1 μL 0.1 N NaOH | 544.504 |
| Initial pH 8.5 | 552.629 |
| 3 hr pH 6.5 | 736.597 |

The results indicate that the polymer is being cleaved and thus decondensing the pDNA. Over 3 hrs time, the pH of the solution decreased from 8.5 to 6.5.

### Example 18

### Condensation of Rh-DNA with polymerized N,N-dimethylformamide and Cleavage with NaOH and stability in 150 mM NaCl

Complexes were made as follows.
Complex I: To NaCl (500 μL of 150 mM), was added RhDNA (5 μL of a 2 μg/μL solution), the solution was vortexed, and the fluorescence intensity measured. MC1015 (8 μg, 4 μL of 2 μg/μL DMF) was added, the solution vortexed, and the fluorescence intensity measured. The fluorescence intensity was measured again after 15 min, 1 hr and 4 hr.
Complex II: To NaCl (500 mL of 150 mM), was added RhDNA (5 μL of a 2 μg/μL solution), the solution was vortexed, and the fluorescence intensity measured. MC1015 (8 μg, 4 RL of 2 μg/μL DMF) was added, the solution was vortexed, and the fluorescence intensity measured. The fluorescence intensity was measured again after 15 min. NaOH was added to the solution and mixed with vortexing. The fluorescence intensity was measured again after 15 min, 1 hr and 4 hr.
Complex III: To NaCl (500 μL of 150 mM), was added RhDNA (5 μL of a 2 μg/RL solution), the solution was vortexed, and the fluorescence intensity measured. MC1015 (8 μg, 4 μL of 2 μg/μL DMF) was added, the solution vortexed, and the fluorescence intensity measured. MC510 (50 μg, 5 μL of a 10 μg/μL solution) was added to the solution with vortexing. The fluorescence intensity was measured again after 15 min, 1 hr and 4 hr.
Complex IV: To NaCl (500 μL of 150 mM), was added RhDNA (5 μL of a 2 μg/μL solution), the solution was vortexed, and the fluorescence intensity measured. MC1015 (8 μg, 4 μL of 2 μg/μL DMF) was added, the solution vortexed, and the fluorescence intensity measured. MC510 (50 μg, 5 FL of a 10 μg/μL solution) was added to the solution with vortexing. The fluorescence intensity was measured again after 15 min. NaOH was added to the solution and mixed with vortexing. The fluorescence intensity was measured again after 15 min, 1 hr and 4 hr.

Results:

| Complex | Fluorescence Intensity (AU) |
|---|---|
| 5 μg RhDNA | 980.009 |
| +8 μg MC1015 (pH5) | 192.373 |
| 15 min (pH5) | 61.978 |
| 1 hr | 40.065 |
| 4 hr | 41.533 |
| 5 μg RhDNA | 980.009 |
| +8 μg MC1015 (pH5) | 204.242 |
| 15 min (pH5) | 98.191 |
| +4 μL 0.1 N NaOH (pH8.5) | 164.544 |
| 1 hr | 207.708 |
| 4 hr | 205.023 |
| 5 μg RhDNA | 501.220 |
| +8 μg MC1015 (pH5) | 90.369 |
| +50 μg MC510 | 218.421 |
| 15 min (pH5) | 172.466 |
| 1 hr | 109.208 |
| 4 hr | 149.865 |
| 5 μg RhDNA | 980.009 |
| +8 μg MC1015 (pH5) | 286.635 |
| +50 μg MC510 (pH6) | 532.596 |
| 15 min | 415.543 |
| +9 μL 0.1 N NaOH (pH8.5) | 373.610 |
| 1 hr | 207.708 |
| 4 hr | 205.023 |

Results indicate that 150 mM NaCl helps stabilize the particle showing less decondensation of the particle when compared to no salt containing systems (example 17).

### Example 19

### Particle Sizing of pDNA complexes with polymerized N,N-dimethylformamide

Particles were formulated as indicated below. Particle solutions were vortexed and measured (Particle Sizer, Brookhaven Instruments).

| | | | Avg. |
|---|---|---|---|
| | | | Count |
| | Volume | Eff. Diam | Rate |
| Particle | (nm) | (nm) | (kcps) |
| pDNA (10 μg) + MC1015 (24 μg)/H₂O | 33.5 | 121.6 | 387.5 |
| (500 μL) | | | |
| pDNA (10 μg) + MC1015 (24 μg)/H₂O | 19.3 | 135.9 | 416.1 |
| (500 μL) 10 min | | | |
| pDNA (10 μg) + MC1015 (24 μg)/NaCl | 29.9 | 166.3 | 538.1 |
| (500 μL, 150 mM) | | | |
| pDNA (10 μg) + MC1015 (24 μg)/NaCl | 21.5 | 178.7 | 558.3 |
| (500 μL, 150 mM) 10 min | | | |
| pDNA (10 μg) + MC1015 (24 μg) + sPLL | 165.2 | 72.7 | 189.6 |
| (70 μg)/H₂O (500 μL) | | | |

### Example 20

### Condensation of polyanions with polymerized N,N-dimethylformamide

To H₂O (500 μL), was added various amounts of Rhodamine labeled polyanions (polyacrylic acid (pAA), succinylated poly-L-lysine (sPLL), and polyaspartic acid (pAsp)), the solutions were mixed, and the fluorescent intensity's were measured on a spectrophotometer (Varian Cary Eclipse Fluorescence Spectrophotometer, Ex=559 nm, Em=576 nm). MC1015 (1 μL of 1 μg/μL DMF) was added, the solution was mixed, and the fluorescence intensity was measured on the spectrophotometer. Subsequent additions of MC1015 were added to the solution until no further decrease in fluorescence intensity was observed. NaOH was added to the solution and mixed with vortexing. The fluorescence intensity was measured again.

| Complex | Fluorescence Intensity (AU) |
|---|---|
| Rh-pAA (3.2 μg) | 160.567 |
| +MC1015 (1 μg) | 81.241 |
| +MC1015 (1 μg) | 71.342 |
| +MC1015 (1 μg) | 65.504 |
| +0.1 N NaOH (5 μL) pH 7.5 | 221.507 |
| Rh-sPLL (40 μg) | 143.895 |
| +MC1015 (1 μg) | 125.608 |
| +MC1015 (1 μg) | 117.313 |
| +MC1015 (1 μg) | 108.850 |
| +MC1015 (1 μg) | 98.686 |
| +MC1015 (1 μg) | 89.425 |
| +MC1015 (1 μg) | 83.965 |
| +0.1 N NaOH (5 μL) pH 7.5 | 136.433 |
| Rh-pAsp (2.1 μg) | 156.158 |
| +MC1015 (1 μg) | 94.460 |
| +MC1015 (1 μg) | 55.636 |
| +MC1015 (1 μg) | 59.008 |
| +0.1 N NaOH (9 μL) pH 7.5 | 120.777 |

Results Indicate that MC1015 Condenses Polyanions and is Labile Under Basic Conditions

The foregoing is considered as illustrative only of the principles of the invention. Furthermore, since numerous modifications and changes will readily occur to those skilled in the art, it is not desired to limit the invention to the exact construction and operation shown and described. Therefore, all suitable modifications and equivalents fall within the scope of the invention.

1. A polycation for delivery of a polyanion to a cell made by the process comprising:
incubating a plurality of formamides in a acidic solution such that at least one imidate is formed.

2. The polycation of claim 1 wherein the polycation condenses nucleic acid.

3. The polycation of claim 1 wherein the polycation consists of a homopolymer.

4. The polycation of claim 3 wherein the homopolymer is formed from the polymerization of formamide monomers selected from the group consisting of: dimethylformamide, dibutylformamide, dimethylthioformamide, and dibutylthioformamide.

5. The polycation of claim 1 wherein the polycation consists of a heteropolymer.

6. The polycation of claim 5 wherein the heteropolymer is formed from the polymerization of formamide monomers selected from the group consisting of: dimethylformamide, dibutylformamide, dimethylthioformamide, and dibutylthioformamide.

7. A polycation formed by the process comprising:
a) incubating in a acidic solution a plurality of formamide monomers of the general structure: 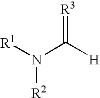
wherein at least one imidate is formed and wherein R¹ and R² independently consist of hydrogen, a primary, secondary, or tertiary carbon in which the substitution is not limited, a methane group in which the substitution is not limited, a methyne group in which the substitution is not limited, or an aromatic or heteroring system, but not a carbonyl group, and R³ consists of a heteroatom.

8. The polycation of claim 7 wherein the polycation condenses nucleic acid.

9. The polycation of claim 7 wherein the polycation consists of a homopolymer.

10. The polycation of claim 9 wherein the homopolymer is formed from the polymerization of formamide monomers selected from the group consisting of: dimethylformamide, dibutylformamide, dimethylthioformamide, and dibutylthioformamide.

11. The polycation of claim 7 wherein the polycation consists of a heteropolymer.

12. The polycation of claim 11 wherein the heteropolymer is formed from the polymerization of formamide monomers selected from the group consisting of: dimethylformamide, dibutylformamide, dimethylthioformamide, and dibutylthioformamide.

13. The polycation of claim 7 wherein R³ is an oxygen atom.

14. The polycation of claim 7 wherein R¹ and R² independently selected from the group consisting of: methyl group and butyl group.

15. The polycation of claim 7 wherein the solution consists of an aqueous solution.

16. The polycation of claim 7 wherein the solution consists of an organic solution.

17. A process for delivering a nucleic acid to a cell comprising:
a) forming a polycation by incubating a plurality of formamides in a acidic solution such that at least one imidate is formed;
b) associating the polycation with the nucleic acid to form a complex; and,
c) contacting the complex with the cell.

18. The process of claim 17 wherein the cell is selected from the group consisting of in vivo cell, in situ cell, and in vitro cell.

19. The process of claim 17 wherein the nucleic acid is selected from the groups consisting of: DNA, plasmid DNA, RNA, double strand DNA, double strand RNA, single strand DNA, single strand RNA, oligonucleotide, siRNA, mRNA, antisense polynucleotide, and antisense polynucleotide.

20. The process of claim 17 further comprising: adding a polyanion to the complex of step b) to form a ternary complex.


SEQUENCE LISTING (8 sequences)

SEQ ID NO: 1 - protein, 10 aa, Human immunodeficiency virus
GRKKRRQRRR

SEQ ID NO: 2 - protein, 16 aa, Drosophila melanogaster
RQIKIWFQNRRMKWKK

SEQ ID NO: 3 - DNA, 21 bp, Photinus pyralis
cuuacgcugaguacuucgatt

SEQ ID NO: 4 - DNA, 21 bp, Photinus pyralis
ucgaaguacucagcguaagtt

SEQ ID NO: 5 - DNA, 20 bp, Aequorea victoria
gacguaaacggccacaagug

SEQ ID NO: 6 - DNA, 21 bp, Aequorea victoria
cgcugcauuugccgguguuca

SEQ ID NO: 7 - DNA, 21 bp, Photinus pyralis
cguacgcggaauacuucgatt

SEQ ID NO: 8 - DNA, 21 bp, Photinus pyralis
ucgaaguauuccgcguacgtt